(19) United States
(12) Patent Application Publication
(10) Pub. No.: US 2025/0161032 A1
(43) Pub. Date: May 22, 2025

(54) ARTIFICIAL POLYMERIC VALVE SYSTEM AND METHODS OF MAKING AND USING THEREOF

(71) Applicants: Arizona Board of Regents on Behalf of The University of Arizona, Tucson, AZ (US); The Research Foundation for The State University of New York, Albany, NY (US)

(72) Inventors: Marvin J. Slepian, Tucson, AZ (US); Danny Bluestein, Stony Brook, NY (US); Brandon J. Kovarovic, Stony Brook, NY (US); Ryan T. Helbock, Stony Brook, NY (US)

(21) Appl. No.: 18/842,656

(22) PCT Filed: Mar. 1, 2023
(86) PCT No.: PCT/US2023/063493

§ 371 Date: Aug. 29, 2024

Related U.S. Application Data
(60) Provisional application No. 63/315,286, filed on Mar. 1, 2022.

(51) Int. Cl. A61F 2/24

(52) U.S. Cl. CPC A61F 2/2418; A61F 2/2415; A61F 2/2476; A61F 2250/0036

(57) ABSTRACT

Artificial polymeric valve systems having a supportive frame embedded in a polymeric sleeve and a plurality of polymeric leaflets forming a continuum with the polymeric sleeve are described herein. The valve systems described can include supportive frames which have at least one non-circular cross-section with polymeric leaflets that are symmetrical or asymmetrical. The leaflets can also have variable thicknesses. Methods of making and using such artificial polymeric valve systems are also described.

### CROSS-REFERENCE TO RELATED APPLICATIONS

This application claims benefit and priority to U.S. Application No. 63/315,286, filed Mar. 1, 2022, and U.S. Application No. 63/373,406, filed Aug. 24, 2022, the disclosures of which are incorporated herein by reference.

### STATEMENT REGARDING FEDERALLY SPONSORED RESEARCH OR DEVELOPMENT

This invention was made with Government Support under Grant No. EB026414 and awarded by the National Institutes of Health. The government has certain rights in the invention.

### FIELD OF THE INVENTION

The invention is generally in the field artificial polymeric valve systems and methods of making and using thereof.

### BACKGROUND OF THE INVENTION

Bicuspid aortic valve (BAV) is the most common congenital heart abnormality (Siu S C, et al., Journal of the American College of Cardiology. 2010; 55(25):2789), characterized by the presence of only two leaflets, as opposed to a normal trileaflet aortic valve (TAV). Most BAVs are formed from the fusion of adjacent aortic valve cusps, with a fibrous seam, i.e. a raphe, typically found along the leaflet fusion junction-considered type 1 and 2 BAVs. BAV type 0 does not have a raphe and is therefore considered “non-malignant” with lower complication rates. Type 1 BAV, which is the most common subtype and is often asymmetrical with one larger leaflet (the fused cusps) and one smaller leaflet, producing a significantly less circular orifice than in TAV anatomy (Shibayama K, et al., J Am Soc Echocardiogr. 2014; 27(11): 1143-52). This less circular orifice anatomy is associated with numerous complications, such as early onset of calcific aortic valve disease (CAVD), aortic stenosis (AS), and a dilated ascending aorta (Lavon K, et al., Annals of biomedical engineering. 2021; 49(12):3310-22; Tchetche D, et al., Circulation: Cardiovascular Interventions. 2019; 12(1):e007107; Yoon S-H, et al., Bicuspid Aortic Valve Morphology and Outcomes After Transcatheter Aortic Valve Replacement. Journal of the American College of Cardiology. 2020; 76(9):1018-30).

CAVD is characterized by the development of calcium deposits on the leaflets. Disease progression increases the overall leaflet stiffness and severely restricts their range of motion. Further exacerbating the anatomical asymmetry, these calcifications typically develop with the raphe or the fused leaflets containing the majority of the bulking mass. During CAVD progression, BAVs are more susceptible to early and increased severity aortic stenosis (AS); 33% of individuals with BAV are expected to develop moderate to severe AS (Rodrigues I, et al., Cardiol Young. 2017; 27(3):518-29. Epub 2016/12/13), and BAV patients comprise approximately 45-49% of all AS cases (Abrams J. The Aortic Valve by Mano Thubrikar Crc Press, Inc., Boca Raton (1990) 221 pages, illustrated, $97.50 ISBN: 0-8493-4771-8. Clinical Cardiology. 1991; 14(4):364a-5). The reduced aortic orifice area results in elevated jet velocity with high instability (Yoganathan A P. Eur Heart J. 1988; 9 Suppl E:13-7), and the less circular orifice shape of the BAV can cause the jet to be directed to the wall of the ascending aorta. This produces increased wall shear stress (WSS), which is significantly higher than TAVs, causing thinning and degradation of the endothelial layer, resulting in aneurysm formation and potential rupture or aortic dissection (Bollache E, et al., J Thorac Cardiovasc Surg. 2018; 156(6):2112-20.e2; Meierhofer C, et al., European Heart Journal—Cardiovascular Imaging. 2012; 14(8):797-804).

The minimally invasive transcatheter aortic valve replacement (TAVR) procedure, in which a bioprosthetic valve attached to a stent frame is introduced via catheter and anchored within the diseased native aortic valve, has become a popular alternative to traditional surgical aortic valve replacement (SAVR), in which the patient undergoes open-heart prosthetic valve placement. The invasive nature of SAVR significantly increases morbidity in high-risk patients who are incapable of enduring such a procedure, leaving TAVR as the primary life-saving treatment. Additionally, TAVR dramatically reduces procedural time and costs, as well as recovery time for patients. In 2017 TAVR gained FDA approval for operable intermediate-risk patients (Rotman O M, et al., Expert Rev Med Devices. 2018; 15(11):771-91), and its use is rapidly expanding to younger and low-risk patients (Durko A P, et al., Trends Cardiovasc Med. 2018; 28(3): 174-83).

Current TAVR devices on the market are designed specifically for calcific trileaflet aortic valve (TAV) stenosis, but have also been used “off-label” to treat inoperable calcific bicuspid aortic valve (BAV) stenosis as the only therapeutic option available. Due to the constrained, less circular and eccentric anatomy and heavily calcified asymmetrical leaflets of BAV (Tchetche D, et al., Circulation: Cardiovascular Interventions. 2019; 12(1):e007107), increased severity and rates of intra- and postoperative complications have arisen, such as poor anchoring and elliptical deployment (Anam S B, et al., J Cardiovasc Transi Res. 2021, 10.1007/s12265-021-10191-z; Iannopollo G, et al., Int J Cardiol. 2020; 317:144-51; Lavon K, et al., Medical & biological engineering & computing. 2019; 57(10):2129-43). The latter can lead to incomplete valve expansion, causing higher transvalvular pressure gradients and increased leaflet stresses (Martin C, et al., Annals of biomedical engineering. 2017; 45(2):394-404), lower valve performance, i.e. characterized as effective orifice area (EOA) (Tchetche D, et al., Circulation: Cardiovascular Interventions. 2019; 12(1):e007107), leading to increased cardiac burden, and central and paravalvular regurgitation (Anam S B, et al., J Cardiovasc Transl Res. 2021, 10.1007/s12265-021-10191-z; Lavon K, et al., Medical & biological engineering & computing. 2019; 57(10):2129-43).

Accordingly, there remains a need for artificial polymeric valve systems, which can be used as replacements for defective valves and that can address the issues and challenges with current valves referenced above, particularly with respect to treating BAV patients.

Therefore, it is the object of the present invention to provide new artificial polymeric valve systems with improved designs and performance characteristics.

It is a further object of the present invention to provide methods of making such artificial polymeric valve systems.

It is still a further object of the present invention to provide artificial polymeric valve systems for use in replacements of defective valves in patients in need thereof.

### SUMMARY OF THE INVENTION

Artificial polymeric valve systems and methods of making and using thereof are described herein. In one non-limiting instance, an artificial polymeric valve system includes a supportive frame, such as a stent, which is embedded in a polymeric matrix forming polymeric sleeve and a plurality of polymeric leaflets form a continuum with the polymeric sleeve without sutures, where each of the polymeric leaflets is connected to the polymeric sleeve at an attachment end.

In at least one non-limiting instance, an artificial polymeric valve system includes:
a supportive frame and a plurality of polymeric leaflets;
wherein the supportive frame comprises a plurality of openings, wherein the supportive frame has a long axis and at least one or more cross-sections taken orthogonally to the long axis have a non-circular shape, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end; and
wherein the polymeric leaflets are able to open and close at an operative end, wherein when the plurality of polymeric leaflets is in the closed position, the operative ends of the leaflets abut each other.

In yet another non-limiting instance, an artificial polymeric valve system includes:
a supportive frame and a plurality of polymeric leaflets;
wherein the supportive frame comprises a plurality of openings, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end;
wherein at least one or more of the plurality of polymeric leaflets is asymmetrical to the other polymeric leaflets in the plurality; and
wherein the polymeric leaflets are able to open and close at an operative end, wherein when the plurality of polymeric leaflets is in the closed position, the operative ends of the leaflets abut each other.

In instances where the supportive frame includes at least one or more cross-sections that have a non-circular shape, the non-circular shape can be selected from an elliptical, oval, lemniscate, cardioid, quartic bean curve, or polygonal (having at least 5, 6, 7, 8, 9, 10, 11, or 12 sides) shape; or the non-circular shape includes a fluctuating asymmetry; or the non-circular shape is a copy of the cross-sectional shape of a native valve of a subject. In some instances, optionally the native valve of the subject is a bicuspid aortic valve.

The number of polymeric leaflets which may be present in the artificial polymeric valve systems described is not particularly limited but requires at least two polymeric leaflets. In some instances, the plurality of polymeric leaflets in the valve systems include from two, three, four, five, six, seven, eight, nine, ten, eleven, or twelve polymeric leaflets. In some cases, the number of leaflets present are selected to be 3, 4, or 5 polymeric leaflets. Greater numbers of leaflets may be present, such as up to one hundred leaflets, and may depend on the size of the valve system where small valve systems include less leaflets and large valve systems include more leaflets.

In some instances, the artificial polymeric valve systems include a supportive frame having a non-circular shaped cross-section and the plurality of polymeric leaflets are all symmetrical with respect to the other polymeric leaflets present in the plurality. In some other instances, the artificial polymeric valve systems include a supportive frame having a non-circular shaped cross-section and at least one or more of the plurality of polymeric leaflets is asymmetric with respect to the other polymeric leaflets present in the plurality.

In some instances, the plurality of polymeric leaflets are variable thickness polymeric leaflets. Such variable thickness polymeric leaflets in the valve system can achieve lower functional stresses within the polymeric leaflets and reduce peak stresses at critical cycle time points during operation of the valve.

Various methods of making the artificial polymeric valve systems described above are also described herein. In one non-limiting instance, a method of making the valve system can include the steps of:
(i) deforming a supportive frame, wherein the supportive frame has a long axis and at least one or more cross-sections taken orthogonally to the long axis have a non-circular shape after the supportive frame is deformed;
(ii) placing the deformed supportive frame inside a cavity of a mold in a position suitable to facilitate flow of the polymer around the deformed supportive frame, wherein the mold comprises a top core, a bottom core, and one or more encasement components which encase the top and bottom cores, wherein the top core and the bottom core define a shape to form a plurality of polymeric leaflets in a suitable orientation wherein the plurality of polymeric leaflets are formed optionally in a semi-open position having a zero residual stress conformation, optionally, wherein the shape forming the plurality of polymeric leaflets has a curvilinear profile along a circumferential axis of the polymeric leaflets being formed; and
(iii) introducing at least one polymer into the mold;
(iv) molding or casting the plurality of polymeric leaflets and a polymeric sleeve around the deformed supportive frame to form the artificial polymeric valve system, wherein the plurality of polymeric leaflets and the polymeric sleeve are a continuum and each of the polymeric leaflets are connected to the polymeric sleeve at an attachment end; and
(v) removing the artificial polymeric valve system formed from the mold.

In instances of the above method, the supportive frame includes at least one cross-section with a non-circular shape which can be set by deforming a supportive frame, such as a stent, along a long or major axis of the supportive frame (see FIG. 4). In such instances, the non-circular shape can be selected from an elliptical, lemniscate, cardioid, quartic bean curve, or polygonal (having at least 5, 6, 7, 8, 9, 10, 11, or 12 sides) shape; or the non-circular shape can include a fluctuating asymmetry; or the non-circular shape is a copy of the cross-sectional shape of a native valve of a subject. In some instances, optionally the native valve of the subject is a bicuspid aortic valve.

In yet another non-limiting instance, a method of making the valve system can include the steps of:
(i′) placing a supportive frame inside a cavity of a mold in a position suitable to facilitate flow of the polymer around the supportive frame,
wherein the mold comprises a top core, a bottom core, and one or more encasement components which encase the top and bottom cores,
wherein the top core and the bottom core define a shape to form a plurality of polymeric leaflets in a suitable orientation wherein the plurality of polymeric leaflets are formed preferably in a semi-open position having a zero residual stress conformation,
optionally, wherein the shape forming the polymeric leaflets has a curvilinear profile along a circumferential axis of the polymeric leaflets being formed; and
(ii′) introducing at least one polymer into the mold;
(iii′) molding or casting the plurality of polymeric leaflets and a polymeric sleeve around the supportive frame to form an artificial polymeric valve system,
wherein at least one or more of the plurality of polymeric leaflets formed is asymmetrical to the other polymeric leaflets in the plurality;
wherein the plurality of polymeric leaflets and the polymeric sleeve are a continuum and each of the polymeric leaflets are connected to the polymeric sleeve at an attachment end; and
(iv′) removing the artificial polymeric valve system formed from the mold.

The artificial polymeric valve systems described herein can be used to replace organ valves in an animal subject, such as human. Such organ valves can be diseased, defective, or otherwise compromised valves. Alternatively, the polymeric valve system may be utilized to replace one or more heart valves; or serve as an extracardiac valve, e.g. in the aorta or other arteries. In still other instances, the polymeric valve system is not limited to use only in the heart or structures therein and may be used in veins; or other luminal structures organs, or organ components of the body of a subject. In one non-limiting example, the artificial polymeric valve systems described can be used to replace a bicuspid aortic valve (BAV), the most common congenital heart malformation, characterized by the presence of only two valve leaflets with asymmetrical geometry, resulting in elliptical systolic opening.

In some instances, the artificial polymeric valve systems can be implanted in a subject in need thereof to replace a defective valve by way of a non-limiting exemplary method which includes the steps of:
(a) inserting an artificial polymeric valve system described herein in a crimped, collapsed, or compacted state into the subject in need thereof;
(b) delivering the artificial polymeric valve system to the defective valve in the subject in need thereof;
(c) implanting the artificial polymeric valve system by expanding the crimped, collapsed, or compacted artificial polymeric valve system into an expanded state, which localizes or fixes the artificial polymeric valve system at the defective valve and replaces the function of the defective valve.

In some other instances, the valve system may be used in a method of treating a subject in need thereof, the method including the steps of:
(a′) inserting an artificial polymeric valve system of any one of claims 1-23 in a crimped, collapsed, or compacted state into the subject in need thereof;
(b′) delivering the artificial polymeric valve system to an artery, vein, or luminal structure organ in the subject in need thereof;
(c′) implanting the polymeric valve system by expanding the crimped, collapsed, or compacted artificial polymeric valve system into an expanded state, which localizes or fixes the artificial polymeric valve system at the artery, vein, or luminal structure organ.

### BRIEF DESCRIPTION OF THE DRAWINGS

FIG. 1A shows an illustration of a deployment of an artificial valve system (denoted device), where the deployed leaflet profile of the device (far right) represents the current standard/commercial approach used, where the targeted deployed device, at the completion of deployment, has a circular shape and the leaflet profile of the device is symmetrical.

FIG. 1B shows a non-limiting illustration of a deployment of an artificial polymeric valve system (denoted device), where the deployed device has a non-circular shape and the leaflet profile of the device is symmetrical (far right).

FIG. 1C shows a non-limiting illustration of a deployment of an artificial polymeric valve system (denoted device), where the deployed device has a non-circular shape and the leaflet profile of the device is asymmetrical (far right).

FIG. 2A shows illustrations of leaflet profiles in a deployed artificial valve system having a circular shape and having two or three symmetrical leaflets, which is representative of the current standard/commercial approach used.

FIG. 2B shows non-limiting illustrations of leaflet profiles in a deployed artificial polymeric valve system having non-circular shapes and having two symmetrical leaflets.

FIG. 2C shows non-limiting illustrations of leaflet profiles in a deployed artificial polymeric valve system having non-circular shapes and having two asymmetrical leaflets.

FIG. 3A shows illustrations of leaflet profiles in a deployed artificial valve system having a circular shape and having two, three, four, five, or six symmetrical leaflets, which is representative of the current standard/commercial approach used.

FIG. 3B shows non-limiting illustrations of leaflet profiles in a deployed artificial polymeric valve system having non-circular shapes and having two or four symmetrical leaflets.

FIG. 3C shows non-limiting illustrations of leaflet profiles in a deployed artificial polymeric valve system having non-circular shapes and having two, three, four, five, or six asymmetrical leaflets.

FIG. 4 shows a non-limiting representation of a supportive frame (i.e., a stent) with three cross-sections taken (d1, d2, and d3) orthogonally to the long axis of the frame, where the middle cross-section forms an elliptical shape d2, as shown in the middle far right portion.

FIG. 5A shows a non-limiting illustration of a top view of three polymeric leaflets (100) of an artificial polymeric valve system where each of the polymeric leaflets has variable thickness along the length of the polymeric leaflet.

FIG. 5B shows a non-limiting illustration of thickness variability across an exemplary polymeric leaflet.

FIG. 6 is a non-limiting representation of a mold (200) having a base core (210), encasement components (220), a top core (230), optionally having multiple axial leaflet lock registrations (240) can be placed around a supportive frame (250).

FIG. 7A shows a non-limiting trileaflet design having two identical leaflets, which has asymmetrical sides, and a smaller symmetric leaflet.

FIG. 7B shows the relative displacement of the coaptation region, or surface area of leaflet abutment towards the adjoining aortic wall, by the movement of the dashed line to the right and the effect on the open profile of the leaflets.

FIG. 8A is an exemplary flowchart showing an asymmetric leaflet implementation strategy for replacing, for example, a defective bicuspid aortic valve (BAV) in an in silico representation of the process. (Left) The supportive frame (i.e., stent) is temporarily held in elliptical shape. (Center) The asymmetric polymeric leaflets are cast, and the supportive frame is released to its original circular shape. (Right) The artificial polymeric valve system is deployed in a subject taking on the elliptical shape.

FIG. 8B is an exemplary flowchart showing an asymmetric leaflet implementation strategy for replacing, for example, a defective bicuspid aortic valve (BAV) in an in silico representation of the process. (Left) The supportive frame (i.e., stent) is provided having a circular-shaped cross-section. (Center) The asymmetric polymeric leaflets are cast. (Right) The artificial polymeric valve system is deployed in a subject.

FIG. 9A shows the finite elemental analysis (FEA) modeling of the deployment of the Evolut R 29 stent in patient specific models.

FIG. 9B shows the systolic opening configuration obtained by applying a pressure gradient to the leaflets with finite element analysis (FEA).

FIG. 10 shows a comparison of the peak systolic stent and leaflet configuration after deployment in six patient-specific BAV models of the asymmetric polymeric BAV (AP-BAV) and Evolut R 29 by Medtronic plc, each listing the geometric orifice area (GOA) and effective orifice area (EOA) for each case.

FIG. 11 shows a non-limiting representation of a lemniscate shape.

FIG. 12 shows a non-limiting representation of a cardioid shape.

FIG. 13 shows a non-limiting representation of a quartic bean curve shape.

### DETAILED DESCRIPTION OF THE INVENTION

Artificial polymeric valve systems and methods of making and using thereof are described below.

### I. Definitions

The term “biocompatible,” as used herein, refers to a material, such as a polymer, which performs its desired function when introduced into an organism without inducing significant inflammatory response, immunogenicity, or cytotoxicity to native cells, tissues, or organs.

The term “hemocompatible” refers to a set of properties of a material that allow for contact with flowing blood without causing adverse reactions such as thrombosis, hemolysis, thrombocytopenia, complement activation, bleeding, or inflammation.

The term “mechanically stable” refers to the ability of a material to maintain its integrity over time when subjected to one or more external stresses.

The terms “conformal,” or “conformally coated,” as used herein typically refer to covering a surface topography of an object with a material such that it is completely or effectively covered up to at least about 50%, 60%, 70%, 80%, 90%, 95%, 96%, 97%, 98%, or 99% of the surface area which is intended to be covered by the material without exposure of the underlying material of the object where covered. The conformal coating is considered to be in direct contact, in intimate contiguity, and matches the geometry and contour to the surface the coating is applied to, so as to act as a cover, barrier, or shield or otherwise form a barrier layer preventing exposure of the underlying surface to exogenous contact by at least about 50 to 100%, as compared with no conformal coating.

The term “fluctuating asymmetry (FA)” refers to a non-directional variation between opposing sides, such as left and right sides, of a bilateral shape. Examples typically include unequal proportions of various pairs of human features, such as between the right and left ears, eyes, or thighs but is also noted in other vertebrate species such as the length of bird wings.

The term “subject” refers to either a human or non-human animal.

The term “treating” refers to inhibiting, ameliorating, impeding, alleviating, or relieving a disease, disorder, or condition from occurring in a subject or causing regression of the disease, disorder, and/or condition. Treating the disease or condition includes ameliorating at least one symptom of the particular disease or condition, even if the underlying pathophysiology is not affected.

Numerical ranges disclosed in the present application include, but are not limited to, ranges of temperatures, ranges of pressures, ranges of integers, ranges of times, and ranges of thicknesses, etc. The disclosed ranges of any type, disclose individually each possible number that such a range could reasonably encompass, as well as any sub-ranges and combinations of sub-ranges encompassed therein. For example, disclosure of a thickness range is intended to disclose individually every possible thickness value that such a range could encompass, consistent with the disclosure herein.

Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers or each of the numbers in the series, unless specified otherwise.

### II. Artificial Polymeric Valve Systems

Artificial polymeric valve systems, including polymeric heart valve systems, are described herein. In one non-limiting instance, an artificial polymeric valve system includes a supportive frame, such as a stent, which is embedded in a polymeric matrix forming polymeric sleeve and a plurality of polymeric leaflets form a continuum with the polymeric sleeve without sutures, where each of the polymeric leaflets is connected to the polymeric sleeve at an attachment end. Further, the plurality of polymeric leaflets are able to open and close at an operative end, wherein when the plurality of polymeric leaflets are in a closed position, the operative ends of the polymeric leaflets abut each other; or in some instances may minimally overlap (such as by less than about 3%, 2%, 1%, 0.9%, 0.8%, 0.7%, 0.6%, 0.5%, 0.4%, 0.3%, 0.2%, 0.1% of the surface area of any abutting leaflet(s).

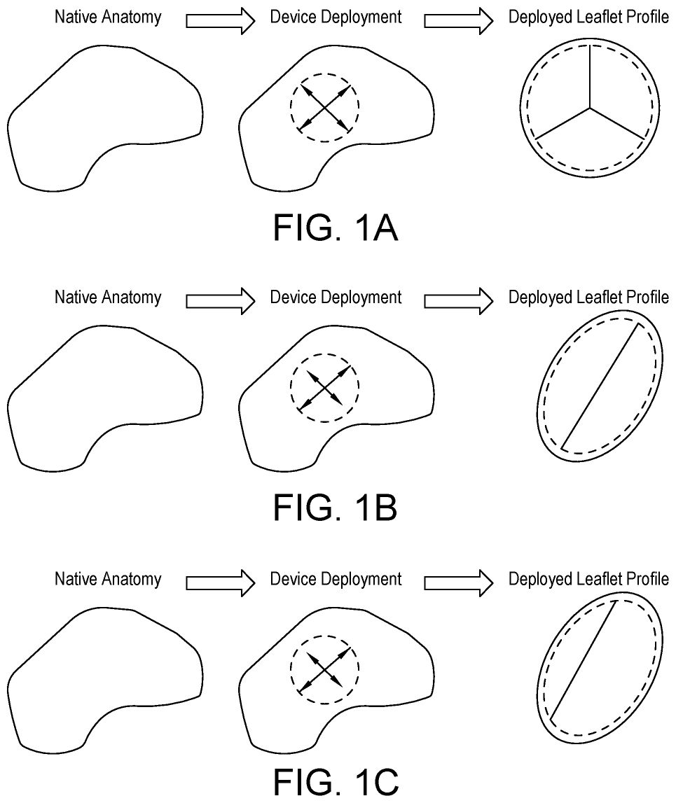

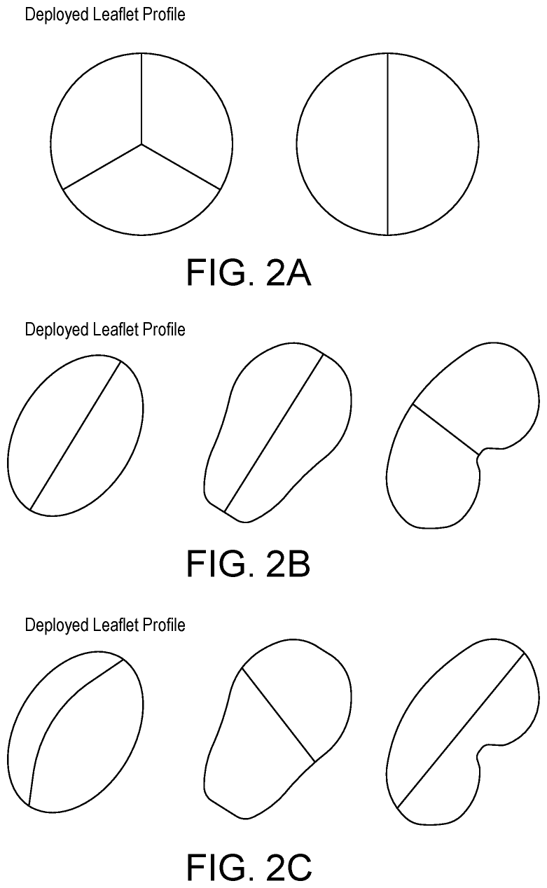

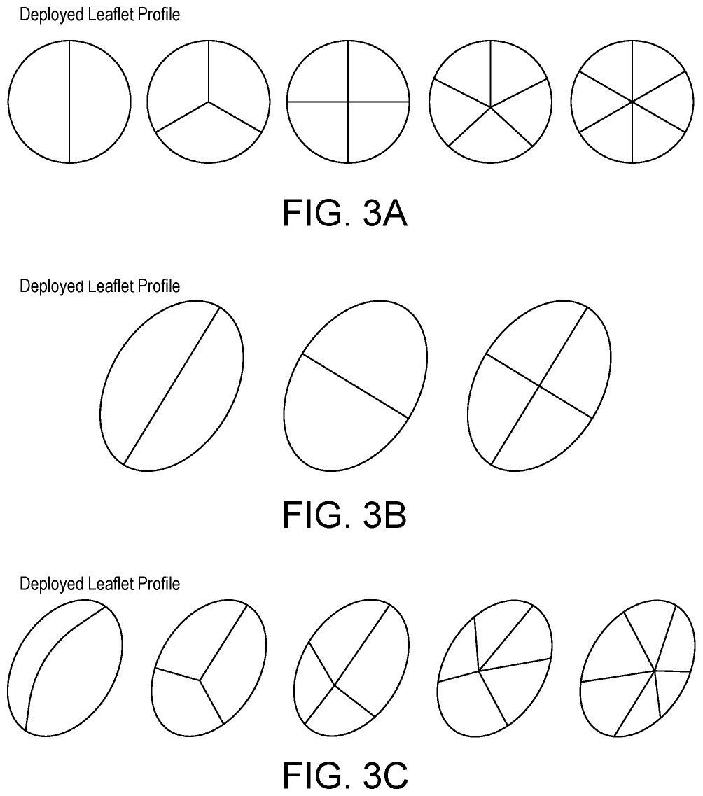

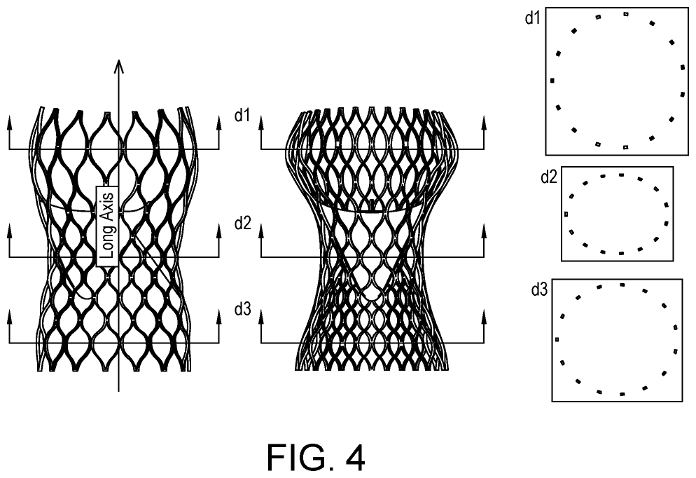

The supportive frame has a long axis and in some instances at least one or more cross-sections taken orthogonally to the long axis have a non-circular shape (see FIGS. 1B, 1C, and FIG. 4). This is in contrast to existing valve systems, which employ stents having circular shapes (see FIG. 1A). In some instances, the plurality of polymeric leaflets are each symmetric with respect to the other leaflets, as may be present (see FIGS. 1B, 2B, and 3B). In some other instances, at least one of the plurality of polymeric leaflets is asymmetric with respect to the other leaflets, as may be present, up to all the polymeric leaflets having a different or unequal surface areas (see FIGS. 1C, 2C, and 3C). This is in contrast to currently known valve systems, which contain symmetrical polymeric leaflets (see FIGS. 2A and 3A).

Accordingly, in one non-limiting instance, an artificial polymeric valve system includes:
a supportive frame and a plurality of polymeric leaflets;
wherein the supportive frame comprises a plurality of openings, wherein the supportive frame has a long axis and at least one or more cross-sections taken orthogonally to the long axis have a non-circular shape, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end; and
wherein the polymeric leaflets are able to open and close at an operative end, wherein when the plurality of polymeric leaflets is in the closed position, the operative ends of the leaflets abut each other.

In another non-limiting instance, an artificial polymeric valve system includes:
a supportive frame and a plurality of polymeric leaflets;
wherein the supportive frame comprises a plurality of openings, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end;
wherein at least one or more of the plurality of polymeric leaflets is asymmetrical to the other polymeric leaflets in the plurality; and
wherein the polymeric leaflets are able to open and close at an operative end, wherein when the plurality of polymeric leaflets is in the closed position, the operative ends of the leaflets abut each other.

In some instances, the polymeric sleeve of the valve systems can include one or more anti-leak flaps and/or the supportive frame can include a plurality of crown supporting struts, a plurality of axial leaflet locking struts, and/or a plurality of optional anchors. In some instances, the artificial polymeric valve systems can have three levels: a crown level, a calcific leaflet level, and a left ventricular outflow track (LVOT) level. The crown supporting struts and axial leaflet locking struts, when present, are preferably not embedded in the polymeric matrix forming the polymeric sleeve.

As described in detail further below, the artificial polymeric valve systems can be formed by placing a supportive frame, such as a stent, in a mold and a suitable polymer can be used to form or cast to the plurality of leaflets of a given design and polymeric sleeve which surrounds the supportive frame or in which the supportive frame is embedded, which can be, for example, an expandable stent.

In instances where the supportive frame includes at least one or more cross-sections that have a non-circular shape, the non-circular shape can be selected from an elliptical, oval, lemniscate, cardioid, quartic bean curve, or polygonal (having at least 5, 6, 7, 8, 9, 10, 11, or 12 sides) shape; or the non-circular shape includes a fluctuating asymmetry; or the non-circular shape is a copy of the cross-sectional shape of a native valve of a subject. Optionally, the native valve of the subject is a bicuspid aortic valve. FIG. 4 shows a non-limiting representation of a supportive frame (e.g., a stent) where the middle cross-section has an elliptical shape.

### A. Components of the Artificial Polymeric Valve System

Various components of the artificial polymeric valve systems are described in detail below.

### 1. Supportive Frame

In some instances, the supportive frame is formed of an armature or scaffold-like construct including a plurality of openings and formed from a plurality of connected struts or wire-like elements, wherein each strut or wire-like element is connected to one or more other struts or wire-like elements via a joint or a node. A joint is understood to refer to a physical frame where the struts connect. Whereas in the case of nodes, which act in a joint or joint-like fashion, in the instance that the supportive frame is formed of a network or mesh of 1D wires or connections, the nodes facilitate opening or otherwise change of shape in the supportive frame with balloon expansion or self-expansion. In some other instances, the armature or scaffold-like construct may be in the form of a cage having tubular or wire-like elements configured in a stent-like configuration. In some instances, the armature or scaffold-like construct is formed having a compressed circumferential/axial profile or can otherwise be formed and subsequently crimped, compacted, or collapsed from its size, as formed, into a smaller circumferential/axial profile suitable for implantation, such as through a delivery catheter. The armature or scaffold-like construct is expandable from its crimped, compacted, or collapsed back up to its size, as formed, or any desired size in between. Preferably, the armature or scaffold-like construct is an expandable stent which is self-expandable or otherwise requires active expansion by balloon expansion, heat expansion, or expansion due to the inherent superelastic properties of the material forming the armature or scaffold-like construct. Expandable stents are plastically deformed with mechanical energy, such as by balloon expansion, into an “opened” or deployed shape. Self-expandable stents can be thermally fixed or shape-set after expansion. Such expandable stents and materials for making such stents are known in the art.

In some other instances, the supportive frame is formed of an armature or scaffold-like construct including a plurality of openings and formed from a plurality of connected struts or wire-like elements, wherein each strut or wire-like element is connected to one or more other struts or wire-like elements in a continuum (i.e., seamlessly). In other words, the supportive frame (i.e., stent) may be a single seamless and continuous structure throughout, whereas in other instances the supportive frame may be formed from more than one piece or component.

In some instances, the maximum diameter of the supportive frame embedded in the polymeric sleeve, when crimped, compacted, or collapsed is about 4, 5, 6, or 7 mm and can be delivered, for example, through an artery, such as the femoral artery. The polymeric sleeve embedded supportive frame once crimped, compacted, or collapsed can be expanded or returned back to its original size and shape when (re)expanded. In some instances, it is better to avoid crimping of the as-formed artificial polymeric valve system until it is time to load it into a delivery catheter, such that the first crimping, compacting, or collapsing of the valve system is during loading into a catheter, or placement or affixing onto a delivery deployment device.

In some cases, once the polymeric valve system is inserted/deployed into a natural valve of the body and fully expanded so there is oversizing, which refers to how much the expanded supportive frame of the valve system is constricted by the surrounding anatomy (i.e., diameter of natural valve orifice). Oversizing can be used to produce a radial force in order to keep the artificial polymeric valve system in a desired position in situ, such as by frictional fit or similar mechanical locking in place. Such constriction is usually proportional to how much radial force the expanded supportive frame of the valve system can exert on the anatomy (e.g., valve orifice) to keep the system in place and avoid migration of the artificial valve system. For the supportive frame, oversizing of about 0 to 40%, about 0 to 30%, about 0 to 20%, about 0 to 15%, about 0 to 10%, or about 0 to 5% and sub-ranges within are acceptable. In some instances, oversizing can be dependent on the radial force that the supportive frame of the system can exert. For example, oversizing of the supportive frame in a relaxed state is considered to be 0%. Oversizing of the supportive frame in the mold, discussed below, can range from about 0 to 8%. Finally, oversizing of the supportive frame, as part of the system, when used in vivo can range from about 5 to 50%, about 5 to 40%, about 5 to 30%, about 5 to 20%, about 5 to 10%, or about 10 to 15%, as well as sub-ranges within. The extent of oversizing can also depend on the anatomical location of implantation of the artificial polymeric valve system.

In some instances, the supportive frame is made of a metal or metal alloy which is known to be suitable for medical implantation. Without limitation, the supportive frame can be formed of self-expanding memory metal alloys, such as NiTiCo, NiTiCr, NiTiCu, NiTiNb, or NiTi (Nitinol) or other known expandable metal/metal alloys, such as spring steels, stainless steel, platinum, tantalum alloys, or cobalt chromium. If actively expandable, the supportive frame can be made of biocompatible metals of particular types, including stainless steel (316L, 304L), cobalt-chromium alloys (L605), nickel-titanium alloy (Nitinol), platinum, and tantalum alloys.

In some instances, the struts or wire-like elements forming the supportive frame can have any suitable dimensions (i.e., length, thickness, radial diameter, etc.). In some cases, the struts or wire-like elements have a tubular shape with a uniform radial tube diameter and the radial thickness ranges from between about 0.2 to 0.6 mm. In some instances, the radial thickness is about 0.3 mm. In some other cases, the struts or wire-like elements are not tubular but have a uniform thickness throughout the struts or wire-like elements and the thickness ranges from between about 0.2 to 0.6 mm. In some other instances, the strut or wire-like elements are curvilinear and can have a variable circumferential thickness along the length of the strut or wire-like element where the thickness of the strut or wire-like element has a 150-250 μm circumferential size and a 250-350 μm radial size, and any sub-ranges or individual values disclosed therein.

In some instances, the struts or wire-like elements of the supportive frame, regardless of shape, are (electro)polished which reduces their thickness prior to the supportive frame being embedded in the polymeric sleeve. Further, (electro)polishing can be performed to remove sharp features that would add stress concentrations, to aid in reducing crimping, compacting, or collapsing strain and resisting plastic deformation during crimping, compacting, or collapsing, as well as increasing the fatigue life/resistance of the formed supportive frame. Additionally, polishing can prepare blood contacting surfaces for better hemocompatibility or provides a more favorable surface morphology to resist thrombus development. In some instances, prior to or following (electro)polishing, the supportive frame may be subjected to sand blasting or chemical etching or other surface modifying method to generate texture and asperities, to the frame surface which can aid in adhesion of the polymer matrix during coating and formation of the polymeric sleeve on the frame; and/or aid in enhancing the durability of adhesion to the frame over use life. In some cases, the supportive frame can be masked before chemical etching or sand blasting to ensure only (electro)polished surface(s) are exposed to blood flow.

The supportive frame may be formed by any suitable method known in the art. Methods of fabrication can include, but are not limited to, laser cutting or etching, laser forming, wire braiding or bending, metal etching, metal vapor deposition, 3-D metal printing, precision machining/CNC, chemical etching, spray coating, sputter coating, powder coating, additive manufacturing, and other art known techniques used to form a stent-like armature or scaffold-like construct suitable for implantation in a subject. As an example, in some instances, a stock material maybe extruded into a cylinder or generated by art known vapor deposition methods for controlled alloy/composite structuring. The cylinder can be, for example, cut with a laser or lathe into a desired shape having a compressed 2D circumferential/axial profile. In forming such supportive frames, full or partial electropolishing may optionally be performed during these deformation(s). Sandblasting and/or electropolishing may also be performed on the supportive frame. Other methods of making such frames, which are implantable stents, are described in U.S. Pat. Nos. 8,715,335; 10,729,824; 10,806,614; 10,874,532; 11,045,297; 11,058,564. In less preferred instances, the supportive frame may be formed of polymer(s), such bio-degradable polymers known in the art.

In some instances, at least some or all of the surfaces of components (e.g., struts) forming the supportive frame include surface texture features, such as micro and/or macro asperities, which impart surface roughness thereon. The surfaces may have morphologies ranging from fully smooth and featureless (e.g. on once face—i.e. adluminal) to others having micro and/or macro asperities (e.g. abluminal face). However, such surface morphologies having asperities are found only on one or more surfaces that are not exposed, for example, to blood flow in order to prevent risk of thrombus formation. Such surface roughness may be imparted by various methods including, but not limited to, plasma etching or chemically etching the supportive frame prior to embedding the frame in a polymeric matrix. Methods and conditions for plasma and chemical etching are known to those skilled in the art. In some other instances, the surfaces of components (i.e., struts) forming the supportive frame can have a suitable primer coating that can chemically bind polymer(s) applied thereto. In one non-limiting example, one or more primer coatings may include a first coat of identical or similar polymer as in the final polymeric sleeve, or other polymers providing adhesive properties, increased durability properties, or additional functionalities, such as providing device radiographic opacity. In some instances, a primer coating may be made of or include silicone polymers.

a. Deformed Supportive Frame

In some instances, the supportive frame includes at least one cross-section with a non-circular shape which can be set by deforming a supportive frame, such as a stent, along a long or major axis of the supportive frame (see FIG. 4). In such instances, the non-circular shape can be selected from an elliptical, oval, lemniscate, cardioid, quartic bean curve, or polygonal (having at least 5, 6, 7, 8, 9, 10, 11, or 12 sides) shape; or the non-circular shape can include a fluctuating asymmetry; or the non-circular shape is a copy of the cross-sectional shape of a native valve of a subject. Optionally, the native valve of the subject is a bicuspid aortic valve.

In some instances, the supportive frame includes at least one cross-section with an elliptical shape. For example, the cross-section can be symmetric about its major and minor axes.

In some instances, the supportive frame includes at least one cross-section with an oval shape. For example, the cross-section can have the shape of an elongated circle.

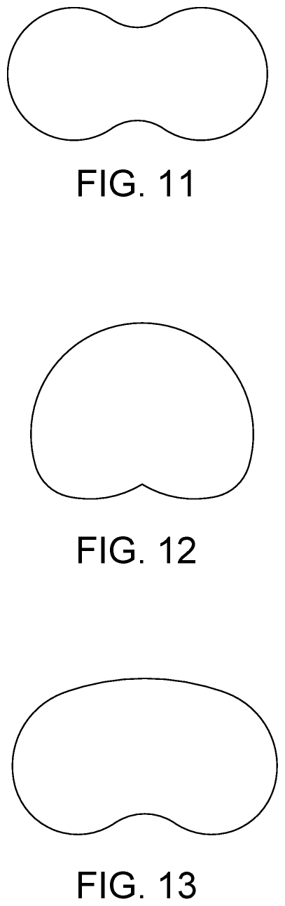

In some instances, the supportive frame includes at least one cross-section with a lemniscate shape. For example, the cross-section can have the shape of a figure eight (8), e.g., as if a circle, an ellipse, or an oval is pinched about its center. A non-limiting example of a lemniscate shape is shown in FIG. 11.

In some instances, the supportive frame includes at least one cross-section with a cardioid shape. For example, the cross-section can have the shape of a heart, e.g., as if a circle, an ellipse, or an oval is pinched about point located on its perimeter. A non-limiting example of a cardioid shape is shown in FIG. 12.

In some instances, the supportive frame includes at least one cross-section with a quartic bean curve shape. For example, the cross-section can have the shape of a kidney bean, e.g., as an ellipse or an oval has a curved depression in a region about its center. A non-limiting example of a quartic bean curve shape is shown in FIG. 13.

The non-circular shape can be imparted on a supportive frame by way of subjecting the frame to a deformation process, which can include a single or multiple deforming steps. It is generally sought to avoid damaging the structural integrity of the supportive frame during such deforming process(es). The undeformed supportive frame may be formed using art known techniques, as described above, or may be obtained from commercial sources and subsequently deformed, as needed. In some instances, a deformed supportive frame having a desired non-circular shaped cross-section can be fabricated directly. In one non-limiting instance, the undeformed supportive frame is subjected to a deformation step to impart a non-circular shape to at least a portion or cross-section of the supportive frame.

In some instances, where the non-circular shape is elliptical, the ellipse shape may be defined based on an ellipticity index. The ellipticity index is a calculation based on the maximal diameter of the ellipse along its longest direction divided by the maximal diameter perpendicular to the longer diameter. For instance, a perfect circle would have an ellipticity index of 1. In some instances, the ellipticity index of the elliptical cross-sectional shape of the artificial polymeric valve systems is greater than 1, such as about 1.1, 1.2, 1.3, or greater.

In some instances, the non-circular shape can refer to an eccentric shape, where eccentric is understood by the skilled person to refer to a shape having an axis that is not centrally located. In such instances, the non-circular shape, or non-circularity, may be defined based its eccentricity. In general, eccentricity can be calculated in the same manner as ellipticity index. However, for more complex shapes, central moments and the image centroid can be utilized. In some instances, the eccentricity of the artificial polymeric valve systems is about 1, 1.1, 1.2, 1.3, 1.4, 1.5, 1.6, or 1.7. Other methods of calculating non-circularity include: compactness, which can be calculated as the ratio of four times 71 times the cross-sectional area to the perimeter length squared; roundness, which can be calculated as the ratio of four times iT times the cross-sectional area to the convex perimeter length squared; convexity, which can be calculated as the ratio of the convex perimeter length to the actual perimeter length or with radial distance measured from the cross-sectional centroid. For example, the non-circular cross-section can have a compactness ranging from 0 to 1. For example, the non-circular cross-section can have a roundness ranging from 0 to 1. For example, the non-circular cross-section can have a convexity ranging from 0 to 1.

In some instances, an approach for selecting and forming a non-circular cross-sectional shape to suit the particular geometry of a defective or diseased valve in a patient involves emulating an actual asymmetry present in a cross-sectional image(s) of the diseased or defective valve in the patient. In some instances, the diseased or defective valve is a bicuspid aortic valve. This represents a personalized and customized complex shape. Such non-circular shape may be determined utilizing a range of different techniques including, but not limited to, echocardiographic, X-ray, computer tomography (CT) or magnetic resonance imaging (MRI). In some instances, the non-circular shape is obtained by such imaging and a copy of the cross-section of the natural or native valve shape can scaled to be an overall net cross-sectional area reduction, such as ranging from 90% 99.9% of the native cross-sectional area, which can be imparted in manufacture to the supportive frame shape, as needed. Upon self, balloon, or thermal expansion the shape may return to the native or natural shape, i.e 100% of the native cross-sectional area, or be further expanded to be larger than the native shape, such as between greater than 100% but less than 200% of the native cross-sectional area. However, even when the shape is expanded to be greater than the native cross-sectional area, the particular native geometry of the imaged native valve is generally maintained.

b. Optional Supportive Frame Components

The supportive frame may include optional components including one or more anchors, one or more indentations, and/or one or more openings, and these are preferably not covered/coated by any polymer or polymeric matrix.

In some instances, one or more anchors which may form part of the strut or wire-like elements of the supportive frame are located on extreme ends of struts present on the supportive frame. The anchors form part of the supportive frame and can be formed during the manufacture of the frame.

The anchors may have any suitable size and shape. In some instances, the anchors form square, rectangular, circular, or oval ring shapes. The anchors, in some instances, are designed to interface the supportive frame into a mold and hold it and constrain the supportive frame during the molding process to embed it in the polymeric sleeve. The anchors, in some other instances, can serve as imaging marks during deployment of the polymeric valve system when the anchors are marked with a radiopaque material or metal. Exemplary imaging marker materials can include, but are not limited to, gold, tantalum, or platinum-iridium. It is possible to mechanically connect a piece of the imaging marker material to specific regions in the supportive frame, which can be designed specifically for that purpose (e.g., leaving a void for placing the marker). In such instances, when used as imaging marks, the anchors designate the top and bottom of the artificial polymeric valve system and can be used to find the location of the polymeric leaflets.

In some instances, one or more indentations and/or one or more openings may form part of the strut or wire-like elements of the supportive frame. The indentations can be used for mechanically locking the polymeric sleeve to the supportive frame. Such indentation or openings can be spread along the supportive frame so as to minimize separation and sliding of the polymer on the struts or wire-like elements of the supportive frame during crimping/compacting/collapsing of the embedded supportive frame, as well as during deployment (i.e., implantation in a subject) and expanding the supportive frame of the system to localize/implant it in a valve. The indentations or openings can be placed in the middle of the struts or wire-like elements of the supportive frame, a region where deformation of the strut is believed to be relatively small, such that the indentation would not impair the mechanical stability of the stent. The indentations or openings can be formed during or following the manufacture of the supportive frame and prior to embedding the frame in the polymeric sleeve. In some instances, the indentations can have a depth of about 10 to 100 μm or a depth of about 10, 20, 30, 40, 50, 60, 70, 80, 90, or 100 μm, in relation to a non-indented portion of the struts or wire-like elements.

### 2. Polymeric Sleeve

As described above, the supportive frame is embedded in a polymeric matrix that forms a polymeric sleeve which typically enmeshes and encases all or at least a large portion (i.e., greater than 90% of the surface) of the supportive frame. In some instances, at least portions of the supportive frame are not embedded or otherwise coated with any polymeric matrix or sleeve.

In some instances, the thickness of the embedding polymeric matrix forming the polymeric sleeve can have any suitable thickness thereon. The thickness of the extraluminal polymeric matrix material covering the supportive frame can, for example, when formed by compression molding or other molding techniques have a thickness in a range from about 0 to 500 microns in the circumferential direction and/or a thickness ranging from about 0 to 400 microns in the radial direction of the struts or wire-like elements of the supportive frame. In some instances, an average thickness of about 400 microns in the circumferential direction (i.e., about 200 microns are on each side of the strut) is present. A thickness value of zero, as noted above, can occur in certain cases where the supportive frame is in contact with a mold surface during the embedding of the frame in the polymeric supportive sleeve and some regions of exposed supportive frame could have no or about zero thickness of polymeric matrix thereon. In other instances, where the supportive frame is embedded in a polymeric matrix material by, for example, dip coating the frame into polymer solution, the encapsulation thickness of the struts or wire-like elements of the supportive frame is expected to be smaller with a thickness ranging from about 0 to 200 microns in the circumferential and/or radial directions.

The polymeric sleeve can be formed of any suitable biocompatible, hemocompatible, and mechanically-stable polymer. In preferred instances, the polymeric material will have elastomeric properties allowing for expansion and reconfiguration following deployment, once situated in its deployment location. The polymeric sleeve should not separate from the supportive frame in use and over time. Such suitable polymers are art known for use in medical implants, and possible blends of such polymers are also contemplated.

In some instances, the surface(s) on the outer face of the polymeric sleeve can be made rougher than the surface of the inner surface of the polymeric sleeve (valve lumen) in order to allow for better adhesion and integration of the outer, i.e., ectoluminal surface, to the native tissue in which the valve system is being implanted on.

Without particular limitation, exemplary polymers which can be used to form the polymeric sleeve during molding include thermoplastic polymers/elastomers, thermoset polymers, thermally crosslinkable polymers, elastomeric polymer biomaterials, polymers containing polysulfone(s), polyurethanes, silicones, PTFE, crosslinked poly(styrene-isobutylenese-styrene) (xSIBS), and poly(styrene-isobutylene-styrene) (SIBS). Thermoplastic elastomers (TPEs) can be formed of or contain polymyrcene, polymenthide, and poly(ε-decalactone). Elastomeric biomaterials can include, for example, silicones, thermoplastic elastomers, polyolefins, polydiene elastomers, poly(vinyl chloride), natural rubbers, heparinized polymers, hydrogels, polypeptides elastomers, polysiloxane-urea elastomers, and polyurethanes. As appropriate, the skilled person is able to select conditions (e.g., temperatures, solvent(s), etc.) needed to form polymer melts or form polymer solutions of the aforementioned to be able to embed the supportive frame during molding, as detailed further below.

In some instances, the polymer material which forms the polymeric sleeve may include one or more fiber reinforcement materials. Such fiber reinforcement materials can be formed of or contain, but are not limited to, poly-alkanes, polyethylene, polytetrafluoroethylene, polyamide, polypropylene, polyethyleneterephthalate, polydimethylsiloxane, polyhydroxyalkanoates, polymethylmethacrylate, silicone, parylene, polydimethylsiloxane, SU-8, liquid crystal polymers, polyurethane, polyetherketones, biodegradable polymers. Exemplary biodegradable polymers can include, without limitation, polylactide (PLA), polyglycolide (PGA), polycaprolactone (PCL), and copolymers or blends thereof. In some instances, the polymeric sleeve may include or be marked with one or more radiopaque materials. Exemplary radiopaque materials which can be added to the above polymers forming the sleeve can include, but are not limited to, stainless steel, gold, tantalum, or platinum-iridium, barium, iodine, and alloys, blends, and mixtures thereof.

a. Gel Paving

In some instances, the outer surface of the polymeric sleeve may be at least partially or completely covered with a conformal gel (i.e., ectoluminal gel paving), which can seal gaps that may form around the outer sleeve of the artificial polymeric valve system post deployment into a valve.

In some instances, the outer surface of the polymeric sleeve is enveloped with a desiccated hydrogel coating. Post-deployment into a defective valve in a subject, the desiccated gel is subject to rehydration in situ which will result in gradual swelling of the gel which will form a thicker, outer facing, gel layer, which is free to swell filling any irregular gaps between the polymeric valve system and valve wall. Gel swelling may be limited by: constraint of the gel by the supportive frame, the underlying polymeric sleeve, and/or the minimal swollen thickness of the desiccated hydrogel layer. However, it is believed that sealing and regional efficacy of leak resistance will be increased following by using such conformal gel paving on the polymeric sleeve of the system.

Exemplary gel paving materials can include, but are not limited to, polymeric hydrogels derived from diacrylate poly(ethylene) glycol (PEG) macromers, which can serve as hemocompatible hydrogel networks, along with macromers incorporating oligocarbonate units. Incorporation of oligocarbonate flanks on PEG before acrylation is able to extend gel durability. In some other instances, non-degradable hydrogel alternatives can be employed, which do not contain degradable units in the copolymer.

Gel paving hydrogels, which often contain about 90% water, can be desiccated, with gels shrinking to an extremely low profile to allow crimping and compaction for valve system deployment. Once deployed, with unsheathing, the hydrogels will gradually re-swell (typically in about 2 to 10 min, with up to a 2 to 9-fold increase in volume), regenerating the hydrogel network to efficiently fill-in irregular-shaped gaps and seal the valve system to the surrounding valve, such as aortic valve, preventing leakage.

In some cases, a hydrogel paving material is placed on the polymeric sleeve outer surface by first enveloping a mesh with the material that will be integral to the polymeric sleeve. This enmeshment improves durable bonding, adhesion and resistance to peel, despite crimping and re-expansion of the valve system during deployment.

In some instances, desiccated hydrogel is applied on the outer surface of the polymeric sleeve in a range from between about 0.5 to 5 mm. The gel paving material will swell to fill gaps, many of which may be of varying sizes and shapes.

### 3. Polymeric Leaflets

The valve mechanism of the artificial polymeric valve systems described herein include a plurality of individual polymeric leaflets, where the polymeric leaflets form a continuum with the polymeric sleeve. In other words, the polymeric leaflets are connected directly to the polymeric sleeve and are not separately attached to the sleeve, such as by suturing or other attachment means, where the leaflets and the polymeric sleeve are continuous and do not ever form discrete components from one another.

The suture-less feature of the polymeric leaflets of the system can be achieved, for example, by forming the plurality of polymeric leaflets and polymeric sleeve (onto the supportive frame) at the same time during the molding process (i.e., single manufacturing step) whereby they form a single polymeric component containing both the polymeric leaflets and polymeric sleeve. In less preferred instances, polymeric leaflets may be formed/molded as a first component and then the leaflets and a supportive frame are dip coated into a suitable polymer melt or solution to form the polymeric sleeve. During such instances, the polymer melt or solution forms the polymeric sleeve over the supportive frame and simultaneously attaches to the polymeric leaflets in the mold. Thus, the leaflets become a continuous inseparable part of the formed polymeric sleeve with smooth transitions forming a “continuum.” The plurality of polymeric leaflets which can be formed by molding can be made of the same or a different polymer from which the polymeric sleeve is formed. In most instances, the polymeric leaflets and polymeric sleeve are formed of the same polymer/polymeric matrix.

Exemplary polymers which can be used to form the polymeric leaflets include, but are not limited to, thermoplastic polymers/elastomers, thermoset polymers, thermally crosslinkable polymers, elastomeric polymer biomaterials, polymers containing polysulfone(s), polyurethanes, silicones, PTFE, crosslinked poly(styrene-isobutylenese-styrene) (xSIBS), and poly(styrene-isobutylene-styrene) (SIBS). Thermoplastic elastomers (TPEs) can be formed of or contain polymyrcene, polymenthide, and poly(ε-decalactone). Elastomeric biomaterials can include, for example, silicones, thermoplastic elastomers, polyolefins, polydiene elastomers, poly(vinyl chloride), natural rubbers, heparinized polymers, hydrogels, polypeptides elastomers, polysiloxane-urea elastomers, and polyurethanes. As appropriate, the skilled person would be able to select conditions (e.g., temperatures, solvent(s), etc.) needed to form polymer melts or form polymer solutions of the aforementioned to be able to form the plurality of leaflets during molding, as detailed further below. In some instances, thermoplastic polymers can be chosen when instances of dip coating are used to form the systems described and thermoset polymers can be chosen when instances of molding are used to form the systems described. In still other instances, a combination of both dip coating of the polymeric sleeve and to attach them to previously molded polymeric leaflets can be used.

In some instances, the polymeric leaflets may include or be marked with one or more radiopaque materials. Exemplary radiopaque materials which can be added to the above polymers forming the polymeric leaflets can include, but are not limited to stainless steel, gold, tantalum, or platinum-iridium, barium, iodine, and alloys, blends, and mixtures thereof.

The polymeric leaflets forming the valve can have any suitable dimension, shape, or size needed for purposes of replacing a valve in a subject. Typically, the design dimensions of the valve and polymeric leaflets therein are based on the free or open deployed diameter of the supportive frame when implanted in a subject. In some cases, the valve height and leaflet height can range from about 0.5 to 2 times the diameter of the free or open deployed diameter of the supportive frame when implanted in a subject. The valve or leaflet height can tailored and selected for individualized sizes based on a subject's valve size.

In most instances, the valve system includes polymeric leaflets which each have a curvilinear or wavy profile in a circumferential axis and are preferably formed/molded in a semi-open conformation. Without wishing to be bound by any particular theory, the semi-open profile aids in minimizing both overall stresses (i.e., flexural stresses) in the valve and during formation of the valve in molding. Nevertheless, other leaflet conformations ranging from fully open to fully closed conformations can also be formed/molded. Selection of a particular configuration (e.g., open, semi-open, closed, or variations therein) can be based on the ease of molding the desired profile.

The number of polymeric leaflets which may be present in the artificial polymeric valve systems described is not particularly limited but requires at least two polymeric leaflets. In some instances, the plurality of polymeric leaflets in the valve systems include from two, three, four, five, six, seven, eight, nine, ten, eleven, or twelve polymeric leaflets. In some cases, the number of leaflets present are selected to be 3, 4, or 5 polymeric leaflets. Greater numbers of leaflets may be present, such as up to one hundred leaflets, and may depend on the size of the valve system where small valve systems include less leaflets and large valve systems include more leaflets.

a. Symmetry of Polymeric Leaflets

In some instances, the artificial polymeric valve systems include a supportive frame having a non-circular shaped cross-section and the plurality of polymeric leaflets are all symmetrical with respect to the other polymeric leaflets present in the plurality (see FIGS. 2B and 3B).

In some other instances, the artificial polymeric valve systems include a supportive frame having a non-circular shaped cross-section and at least one or more of the plurality of polymeric leaflets is asymmetric with respect to the other polymeric leaflets present in the plurality (see FIGS. 2C and 3C). In some instances, each of the polymeric leaflets is asymmetrical to all of the other polymeric leaflets in the plurality. In still other instances, some subset(s) (such as a pair(s) of leaflets) of the plurality of polymeric leaflets may be symmetrical to each other but these subset(s) are not symmetrical to other subset(s) present. For instance, in a valve system with four leaflets, two of the leaflets may be equivalent (i.e., having the same size, surface area, and shape) and the other two leaflets may be equivalent (i.e., having the same size, surface area, and shape) but where the first pair and the second pair of leaflets are not equivalent (i.e., having a different size, surface area, and/or shape), where this is a non-limiting example of mixed symmetrical and asymmetrical leaflets but is overall considered an example of asymmetry in the valve system.

b. Variable Thickness

Without being bound to any particular theory, in some instances, the plurality of polymeric leaflets are variable thickness polymeric leaflets. Such variable thickness polymeric leaflets in the valve system can achieve lower functional stresses within the polymeric leaflets and reduce peak stresses at critical cycle time points during operation of the valve.

The particular pattern of thicknesses within each polymeric leaflet can be determined by mapping the time-dependent principal stress distribution of a model of the polymeric leaflets. In a first step, a model is formed and moved through a typical valve operation cycle to determine or estimate the locations and relative amounts, e.g., high versus low, of stress in each location throughout a typical cycle for all or one or more regions within the polymeric leaflet. The relative amounts of stress that can be analyzed correspond with the bending, twisting, stretching, or other motions which can occur during a typical operation cycle. Based on the mapping of such stresses, the particular locations and relative thicknesses for the leaflets can be selected to reduce or minimize stress during cycles of use. Interactive finite element analysis (FEA) can be used to produce/model the aforementioned time-dependent principal stress distribution. A model of the polymeric leaflets can refer to a physical model formed from a material that is the same as or different from the material of the final leaflets. Optionally, the model is a digital model, such as one created using software, which can be digitally manipulated to determine or estimate the relative stresses and their locations during a typical diastole and systole cardiac cycle.

Thus, the polymeric leaflets can each independently have non-uniform thicknesses throughout. In some instances, the variable thickness is understood by considering a center line of symmetry of each of the polymeric leaflets, where in a given cross-section each of the leaflets has at least two or more thicknesses. In some instances, the polymeric leaflets each independently have a non-uniform (i.e., variable) thickness across any cross-sectional direction of the leaflet. In other words, the polymeric leaflets have a variable thickness profile which is not uniform across any of the leaflet's circumferential cross-sections, and without any preferential directionality in the thickness within such cross-sections. Such variable thickness profiles of the leaflets can lead to improved hemodynanics, flexibility, and enhanced or high durability of the polymeric valve. In some instances, enhanced or high durability can be defined based on the FDA ISO 5840 (2013) guidelines which defines that such a valve system, as described herein, would survive at least 200 million cycles or more of rapid valve opening and closing (in a special durability tester)—equivalent to about 5 years operation in a subject (patient). In some instances, the polymeric valve system described can perform up to or at least 1 billion cycles, and potentially more cycles going on (equivalent to about 25 years in a subject). The artificial polymeric valve systems described, and methods of use thereof, can provide valve replacements with durability exceeding current standards for valve testing and durability.

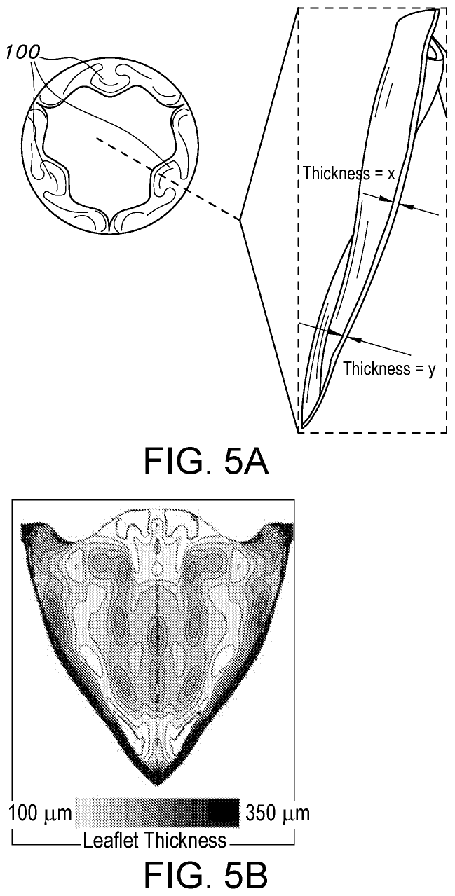

In some non-limiting instances, the thickness at one position of a polymeric leaflet will differ from the thickness at another/different position on the same leaflet. The thicknesses across a longitudinal cross-section of the leaflet may vary with a specified slope, such that the thickness decreases or increases in a given direction of the cross-section. For example, as shown in FIG. 5A, thickness “x” at a first position of a cross-section of an exemplary leaflet 100 is greater than the thickness “y” at a second position. FIG. 5B shows a representation of thickness variability present in an exemplary polymeric leaflet. In still other instances, the thickness from any given position on the polymeric leaflets varies as one moves radially out in any direction to another position.

In some instances, the polymeric leaflets, which are connected to the polymeric sleeve at an attachment end, have a thickness which is greatest at the attachment end to the sleeve and which decreases with distance away from the attachment end in any direction taken until a terminal edge of the leaflet is reached. In other words, the polymeric leaflets may have thicknesses that become thinner moving out from the attachment edge. In some instances, a maximum thickness of any portion of the polymeric leaflets can range from between about 200 to 600 μm or about 400 to 500 μm, as well as sub-ranges within. The maximum thickness may be dictated by the volume of the leaflets which should be reduced for ease of crimping/compacting/collapsing the formed valve system and for effective reduction of the overall stresses on the system. In some instances, a minimum thickness of any portion of the polymeric leaflets can range from between about 50 to 200 μm, about 50 to 150 μm, or about 50 to 100 μm, as well as sub-ranges within. In still other instances, any portion of the polymeric leaflets have a variable and non-uniform thickness can have thickness ranging from between about 50 to 600 μm. The desired thicknesses of any part or portion of each of the respective leaflets can be controlled by way of the molding technique and the lowest limit of capable thickness which can be achieved.

Without wishing to be bound by any particular theory a benefit of variable thickness in the polymeric leaflets is in achieving a maximum peak von Mises stress which is at or below about 30%, 35%, 25%, 20%, 15%, or 10% of the yield stress of the polymer it is composed from, as well as to provide lower and more uniform stress distribution and reducing bending stresses during an entire operation cycle of the valve. Thus, polymeric leaflets with variable thickness can exhibit lower overall stresses while also providing an overall reduction in the volume of material used in forming the leaflets. This can also benefit the ability to crimp/compact/collapse the artificial polymeric valve system due to the decreased volume. Reduction in the polymer volume within the polymeric leaflets can help reduce the size of the valve system for easing delivery of the valve to a subject. In some instances, use of variable thickness polymeric leaflets includes a reduction in peak principal stress (absolute) at or below about +1.2 MPa and above about −1.0 MPa and a reduction in von Mises stresses at below about 1 MPa, with a lower, more uniform stress distribution during peak diastole.

The variable thickness in the polymeric leaflets can be achieved by way of compression or injection molding, where two mating mold parts define the variable thickness throughout the entire leaflet surface being molded. Details of the manufacture of such variable thickness polymeric leaflets is provided below.

Lastly, it is noted that all biological tissue-based valves, which are used in current valve replacements have a roughly uniform thickness because these tissues are harvested from animals and cannot incorporate variable thickness therein.

### 4. Anti-Leak Flap

The polymeric valve systems described, such as the polymeric heart valve systems described herein, optionally contain one or more anti-leak flaps. The one or more anti-leak flaps may be present in the valve system at a suitable position such that when the system is implanted, the anti-leak flaps are present at or near the natural and/or calcific leaflets of the subject. In one non-limiting example, when the artificial polymeric valve system is implanted in a cardiac valve (e.g., aortic valve), the anti-leak flap can be located adjacent to the sinus, native calcific leaflets, and left ventricular outflow track of the heart valve.

In some instances, the one or more anti-leak flaps include a plurality of optional slits which can improve flexibility and the ability of the anti-leak flap(s) to cover or abut against the native leaflets of the valve.

The one or more anti-leak flaps can be formed by molding and can be made of the same or a different polymer from which the polymeric sleeve and/or leaflets is formed. In most instances, the one or more anti-leak flaps, polymeric leaflets, and polymeric sleeve are formed of the same polymer/polymeric matrix. Exemplary polymers which can be used to form the one or more anti-leak flaps include, but are not limited to, thermoplastic polymers/elastomers, thermoset polymers, thermally crosslinkable polymers, elastomeric polymer biomaterials, polymers containing polysulfone(s), polyurethanes, silicones, PTFE, crosslinked poly(styrene-isobutylenese-styrene) (xSIBS), and poly(styrene-isobutylene-styrene) (SIBS). Thermoplastic elastomers (TPEs) can be formed of or contain polymyrcene, polymenthide, and poly(F-decalactone). Elastomeric biomaterials can include, for example, silicones, thermoplastic elastomers, polyolefins, polydiene elastomers, poly(vinyl chloride), natural rubbers, heparinized polymers, hydrogels, polypeptides elastomers, polysiloxane-urea elastomers, and polyurethanes. As appropriate, the skilled person would be able to select conditions (e.g., temperatures, solvent(s), etc.) needed to form polymer melts or form polymer solutions of the aforementioned to be able to form the one or more anti-leak flaps during molding, as detailed further below. Typically, the one or more anti-leak flaps are molded together with the polymeric leaflets and polymeric sleeve onto the supportive frame in a single step.

The one or more anti-leak flaps can have any suitable dimension, shape, or size needed. The length of the anti-leak flap(s) extending from the surface of the polymeric sleeve can range from about 1 to 5 mm. In some instances, the thickness of any portion of the one or more anti-leak flaps can range from between about 0.01 to 100 μm, 30 to 350 μm, or about 30 to 300 μm, where the thickness can be dictated by the molding technique and the lowest limit of capable thickness which can be achieved.

The one or more anti-leak flaps are believed to have the ability to block paravalvular leak (PVL) channels by either covering the entrance to the channel, as it covers or seals gaps between the valve system and the native calcific leaflets or by filling the gaps between the valve system and the native calcific leaflets. PVL channels are complex and highly restricted flow paths due to incomplete sealing between an expanded TAVR device and underlying calcified leaflets and the aortic wall that are driven by large diastolic pressure gradients, creating high velocity jet flows from the native sinuses back into the left ventricular outflow track (LVOT). PVL is often classified by leak severity determined by clinician judgement of the jet velocity and flow.

### III. Methods of Making Artificial Polymeric Valve Systems

Methods of making the artificial polymeric valve systems described above are detailed below.

In one non-limiting instance, a method of making the valve system can include the steps of:
(i) deforming a supportive frame, wherein the supportive frame has a long axis and at least one or more cross-sections taken orthogonally to the long axis have a non-circular shape after the supportive frame is deformed;
(ii) placing the deformed supportive frame inside a cavity of a mold in a position suitable to facilitate flow of the polymer around the deformed supportive frame,
wherein the mold comprises a top core, a bottom core, and one or more encasement components which encase the top and bottom cores,
wherein the top core and the bottom core define a shape to form a plurality of polymeric leaflets in a suitable orientation wherein the plurality of polymeric leaflets are formed optionally in a semi-open position having a zero residual stress conformation,
optionally, wherein the shape forming the plurality of polymeric leaflets has a curvilinear profile along a circumferential axis of the polymeric leaflets being formed; and
(iii) introducing at least one polymer into the mold;
(iv) molding or casting the plurality of polymeric leaflets and a polymeric sleeve around the deformed supportive frame to form the artificial polymeric valve system,
wherein the plurality of polymeric leaflets and the polymeric sleeve are a continuum and each of the polymeric leaflets are connected to the polymeric sleeve at an attachment end; and
(v) removing the artificial polymeric valve system formed from the mold.

In instances of the above method, the supportive frame includes at least one cross-section with a non-circular shape which can be set by deforming a supportive frame, such as a stent, along a long or major axis of the supportive frame (see FIG. 4). In such instances, the non-circular shape can be selected from an elliptical, lemniscate, cardioid, quartic bean curve, or polygonal (having at least 5, 6, 7, 8, 9, 10, 11, or 12 sides) shape; or the non-circular shape can include a fluctuating asymmetry; or the non-circular shape is a copy of the cross-sectional shape of a native valve of a subject. In some instances, optionally the native valve of the subject is a bicuspid aortic valve. The plurality of polymeric leaflets formed in the valve system of the above method may all be symmetrical or asymmetrical with respect to the other polymeric leaflets present in the plurality. In some other instances, at least one or more of the plurality of polymeric leaflets is asymmetric with respect to the other polymeric leaflets present in the plurality, where some of the leaflets may nonetheless be symmetrical to each other.

In yet another non-limiting instance, a method of making the valve system can include the steps of:
(i′) placing a supportive frame inside a cavity of a mold in a position suitable to facilitate flow of the polymer around the supportive frame,
wherein the mold comprises a top core, a bottom core, and one or more encasement components which encase the top and bottom cores,
wherein the top core and the bottom core define a shape to form a plurality of polymeric leaflets in a suitable orientation wherein the plurality of polymeric leaflets are formed preferably in a semi-open position having a zero residual stress conformation,
optionally, wherein the shape forming the polymeric leaflets has a curvilinear profile along a circumferential axis of the polymeric leaflets being formed; and
(ii′) introducing at least one polymer into the mold;
(iii′) molding or casting the plurality of polymeric leaflets and a polymeric sleeve around the supportive frame to form an artificial polymeric valve system,
wherein at least one or more of the plurality of polymeric leaflets formed is asymmetrical to the other polymeric leaflets in the plurality;
wherein the plurality of polymeric leaflets and the polymeric sleeve are a continuum and each of the polymeric leaflets are connected to the polymeric sleeve at an attachment end; and
(iv′) removing the artificial polymeric valve system formed from the mold.

Although the above methods preferably produce polymeric leaflets in a semi-open position (which can reduce stresses, such as flexural stresses), the methods may also be used to produce polymeric leaflets in open or closed positions or any intermediate position. The design of the top and bottom cores can be selected to produce polymeric leaflets of any desired position when these components are created/fabricated.

For the methods above, the number of polymeric leaflets which may be present in the artificial polymeric valve systems formed is not particularly limited but requires at least two polymeric leaflets. In some instances, the plurality of polymeric leaflets in the valve systems formed include from two, three, four, five, six, seven, eight, nine, ten, eleven, or twelve polymeric leaflets. In some cases, the number of leaflets present are selected to be 3, 4, or 5 polymeric leaflets. As described above, the plurality of polymeric leaflets formed by the methods may include variable thickness.

The mold used in the methods described can be a modular mold, where the interior of the mold and the sections that are used to form the artificial polymeric valves are modular (e.g. symmetrically split to three parts in a circumferential axis) to facilitate easy extraction of the fabricated valve system from the molding cavity.

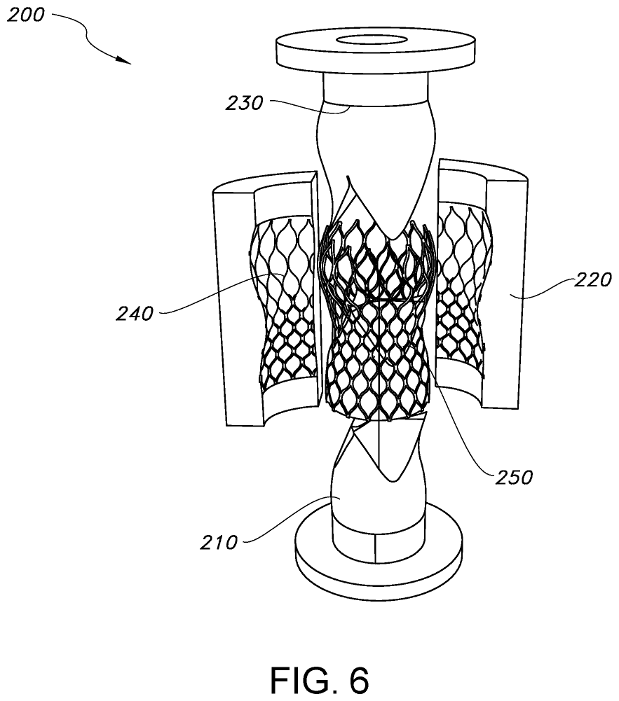

As shown in FIG. 6, a non-limiting mold (200) having a base core (210), encasement components (220), a top core (230), optionally having multiple axial leaflet lock registrations (240) can be placed around a supportive frame (250). Although not illustrated in FIG. 6, the number and size of the encasement components is selected to completely surround and encase the supportive frame (i.e., form a closed mold) during the molding process. The axial leaflet lock registrations can be used to hold the supportive frame, such as at the axial leaflet locking struts (not shown). In instances of the first method, the base core component is designed to deform the supportive frame when the frame is placed thereon and can impart a desired non-circular shape to the supportive frame, which is retained upon completion of the method steps described.

During steps (iii) or (ii′) a polymer is introduced into the mold and the polymeric leaflets and the polymeric sleeve can be simultaneously molded or cast onto the supportive frame. Once the valve system is formed it can be removed during steps (v) or (iv′) from the mold by disassembling the mold parts. Preferably, the mold is designed to not embed any crown supporting struts, axial leaflet locking struts, and any anchors, as may be present, within the polymeric sleeve. In some instances, if the crown supporting struts, axial leaflet locking struts, and any anchors are covered by polymeric sleeve or any excess polymer, this may be removed afterward.

In some instances of the methods, the mold may include one or more vent holes which allow suction of air out of the mold cavity and/or the removal of excess polymer which may be introduced in steps (iii) or (ii′).

In some instances, the mold and components thereof are formed of stainless steel, tool steel, aluminum, or any other suitable metal. The mold and components thereof may be fabricated using art known techniques, such as precision machining/CNC or 3-D metal printing techniques. The design of the mold cavities needed to produce the leaflets and polymeric sleeve on the supportive frame, having any requisite complexity of design, can be achieved in the mold parts using such precision machining/CNC or 3-D metal printing techniques or other precision machining or fabricating means including additive manufacturing. Further, the mold may include sealing elements, such as o-rings, or custom-shape and made sealing elements. For instance, if it is necessary to prevent the polymer from coating a part of the supportive frame, a sealing element with a custom shape can be made for that part of the supportive frame (e.g. from silicone or other suitable materials).

In some instances of the methods, positioning of the supportive frame into the mold cavity (in steps (ii) or (i′)) involves the presence of an annular gap distance from the supportive frame to the outer polymeric sleeve, to be formed thereon, in a size ranging from about 10 to 20 μm. To promote accurate placing of the supportive frame within the desired tolerance, arched segments (e.g. 0.1-1.0 mm thick) may be placed or present in parts of the mold components, such as the encasement components, that protrude from the outer wall of the mold and hold the supportive frame in the desired radial position. In some instances, this allows for full alignment with the artificial polymeric valve system centerline. Such arched segments can be spaced, for example, every 3 to 10 mm (in the long axis). In order to avoid any potential impairment of polymer flow inside the mold, slits can be cut out of the arched segments to for flow of the polymer in between the segments. In some instances, such arched segments may be retractable such that they can retracted from the mold body once the polymer has been cured sufficiently. In still other instances, instead of use of arched segments, these could be replaced with multiple pins that are spaced apart in the circumferential and longitudinal-axial directions of components of the mold. The arched segments should preferably be made of the same material as the mold so that heating and thermal expansion of the mold remains uniform throughout. Nevertheless, use of combinations of various metals or polymers can be used to form the arched segments or mold components that do not experience mechanical loading. As an example, a plunger to apply force/pressure to the polymer can be made of one type of steel (i.e., tool steel) while components with surfaces that are in contact with the valve/stent being molded/cast can be made of a different kind of steel (i.e., stainless steel). Moving parts, such as a plunger, may benefit from being made of harder steel to minimize potential wear and particle leaching.

In some instances of the methods, the mold, prior to step (i)/(ii) or (i′) may be cleaned and prepared (chemically and/or mechanically). In some instances, the mold is heated during steps (iii)/(iv) or (ii′)/(iii′) to a suitable temperature, such as to ensure ease of flow of the polymer through the mold. Such heating temperatures can range from about 100° C. to 350° C., and subranges within. The molding or casting of steps (iv) or (iii′) involve curing the polymer, where after curing is complete (a function of temperature and time) the mold is allowed to cool down, then the mold is opened and the cured artificial polymeric valve is removed. In some instances of the methods, a vacuum can be applied into the mold to remove air bubbles from the polymer introduced into the mold cavity which cured to produce the molded or cast polymer. The ability to produce a vacuum may be integrated into the mold itself. In some instances, when polymer curing is completed, the vacuum (if applied) is released, and the mold is partially opened up while still heated/hot and the internal mold parts that “wrap” the cured polymeric valve are then allowed to cool down, either slowly to room temperature, or quickly by immersion into water.

In some instances of the methods, as the polymer formed by, for example, compression/transfer/injection molding, is cured under heat and pressure, the resulting polymer becomes denser and less porous. Manipulation of the outer surface roughness can assist in adhesion to native tissue of a defective valve. Another potential advantage of such rough surface can be lower friction during crimping/collapsing/compacting of the valve systems for deployment into a subject.

In some instances, the formed polymeric valve system can be carefully extracted from the mold in steps (v) or (iv′) of the methods: by separation from the mold using, for example, a spatula, air pressure, liquid pressure (e.g. isopropyl alcohol), immersion in a solvent (e.g. alcohols, such as ethanol or isopropyl alcohol) optionally with sonication. Following removal of the valve system, the methods described may also involve a further step of removing of any excess polymer from the valve which is not desired, such as by cutting, laser cutting, ultrasonic cutting, thermal cutting, or other cutting means. The methods may additionally involve a further step of cleaning the formed artificial polymeric valve system after removal from the mold in suitable solvent(s), optionally with ultrasonication and then drying the polymeric valve thereafter.

Following the removal step, the methods may further include a step of conformally coating at least part or all of the outer surfaces of the polymeric sleeve with a gel paving material, such as made from a hydrogel. In some instances, the gel paving material may be applied via forming gels as films and wrapping the external surface of the polymeric sleeve and/or polymeric leaflets with the gel paving material, allowing it to dry, thereby constriction boding to the surface. In some other instances, the external surface of the polymeric leaflets and/or polymeric sleeve may be doped with a layer of suitable adhesive to which the gel paving material—either wet or in desiccated form is applied. In still other cases, the external surface of the polymeric leaflets and/or polymeric sleeve may be coated with a photoreactive catalyst—e.g., eosin or riboflavin, and the gel formed as a layer via photocatalysis as described in U.S. Pat. No. 6,290,729 by Slepian. The thickness of the gel paving material, in desiccated form, may range from between about 0.5 to 5 mm.

In some instances of the methods, a plunger or other means of applying pressure may be used when the polymer is introduced in steps (iii) or (ii′). The polymer may be introduced through, for example, an opening in the top core of the mold and a plunger may be used to apply pressure to the polymer to ensure it spreads throughout the cavities of the mold. Suitable pressures, which can be used to flow the polymer during steps (iii) and (iv) and (ii′) and (iii′), can range from between about 0.01 to about 10 tons, and sub-ranges or individual values contained within.

In some instances, the polymeric sleeve formed further includes one or more anti-leak flaps which are formed by using a mold that defines the anti-leak flaps and forms the anti-leak flaps, when the polymer is molded/cast in steps (iv) or (iii′) of the methods described above.

In some cases, one or more releasing agents may be applied to the mold surfaces prior to step (ii) or (i′) in order to facilitate removal of the polymeric valve system from the mold. Suitable mold releasing agents include solvent-based, water-based agents, silicone agents, or resin-based agents, which may be sacrificial or semi-permanent. These agents can be sprayed onto the surfaces at room temperature or with heating (of about 50 to 200° C. or about 100 to 150° C.) in a single or multiple layers. Various types of mold release agents are commercially available and known to those skilled in the art.

In some instances, the molding or casting of the at least one polymer in steps (iv) or (iii′) of the methods is performed by a compression molding process, an injection molding process, a dip coating process, or a transfer molding process. Compression molding, for example, relies on part of the shape setting mold to move and compress the polymer, cause pressure and flow into the other mold cavities. Injection molding, for example, relies on an extruder system to heat and pressurize the polymer, causing it to flow into the mold cavities. Transfer molding, for example, is similar to injection molding, but instead of an extruder generating the pressure, a plunger system is pressurized (compressed) into a cavity of heated polymer and injected into the cavities of the mold. Such processes and conditions for such processes are known to those of ordinary skill in the art. In one non-limiting example of the method, during step (ii) a polymer (e.g., pellets or powder form) is placed into the mold, if compression molding, or into a container adjacent to the mold, if transfer or injection molding is employed, and the mold is heated to a temperature that would make the raw polymer pliable in a fluid gel-like form.

Exemplary polymers, or blends thereof, which can be used to form the polymeric leaflets and polymeric sleeve in the methods described include, but are not limited to, thermoplastic polymers/elastomers, thermoset polymers, thermally crosslinkable polymers, elastomeric polymer biomaterials, polymers containing polysulfone(s), polyurethanes, silicones, PTFE, crosslinked poly(styrene-isobutylenese-styrene) (xSIBS), and poly(styrene-isobutylene-styrene) (SIBS). Thermoplastic elastomers (TPEs) can be formed of or contain polymyrcene, polymenthide, and poly(ε-decalactone). Elastomeric biomaterials can include, for example, silicones, thermoplastic elastomers, polyolefins, polydiene elastomers, poly(vinyl chloride), natural rubbers, heparinized polymers, hydrogels, polypeptides elastomers, polysiloxane-urea elastomers, and polyurethanes. As appropriate, the skilled person would be able to select conditions (i.e., temperatures, solvent(s), etc.) needed to form polymer melts or form polymer solutions of the aforementioned for use in steps (iii) and during molding/casting step (iv) of the first method, or steps (ii′) and (iii′) of the second method described. In some instances, thermoplastic polymers can be chosen when instances of dip coating are used to form the systems described and thermoset polymers can be chosen when instances of compression, transfer, or injection molding are used to form the systems described. In still other instances, a combination of both dip coating of the polymeric sleeve and to attach them to previously molded polymeric leaflets can be used.

Details of the supportive frame, polymeric sleeve, polymeric leaflets, and anti-leak flap(s) which may be formed according to the methods described to provide the artificial polymeric valve systems are given below.

### 1. Supportive Frame

In some instances, the supportive frame used in the methods is formed of an armature or scaffold-like construct formed from a plurality of connected struts, wherein each strut is connected to one or more other struts via a joint or a node. In some other instances of the methods, the supportive frame is formed of an armature or scaffold-like construct including a plurality of openings and formed from a plurality of connected struts or wire-like elements, wherein each strut or wire-like element is connected to one or more other struts or wire-like elements in a continuum (i.e., seamlessly) without joints, where a node for opening or changing the shape of the stent by expansion is present.

In some instances, the armature or scaffold-like construct may be in the form of a cage having tubular or wire-like elements configured in a stent-like configuration. The armature or scaffold-like construct is expandable from its crimped, compacted, or collapsed back up to its size, as formed, or any desired size in between. Preferably, the armature or scaffold-like construct is an expandable stent which is self-expandable or otherwise requires active expansion by balloon expansion, heat expansion, or expansion due to the inherent superelastic properties of the material forming the armature or scaffold-like construct. Expandable stents are plastically deformed with mechanical energy, such as by balloon expansion, into an “opened” or deployed shape. Self-expandable stents can be thermally fixed or shape set after expansion. Such expandable stents and materials for making such stents are known in the art. Suitable materials, dimensions, characteristics, and methods of manufacturing the supportive frame are described above. Further, as previously noted, the supportive frame may include optional components including one or more anchors and/or one or more indentations which are preferably not covered/coated by any polymer or polymeric matrix during or following any steps of the methods above, or where any polymer thereon can be removed after these steps are performed.

a. Deformed Supportive Frame

In instances of the first method, the supportive frame is deformed in the first step to include at least one cross-section with a non-circular shape which can be set by deforming a supportive frame, such as a stent, along a long or major axis of the supportive frame (see FIG. 4). In such instances, the non-circular shape can be selected from an elliptical, lemniscate, cardioid, quartic bean curve, or polygonal (having at least 5, 6, 7, 8, 9, 10, 11, or 12 sides) shape; or the non-circular shape can include a fluctuating asymmetry; or the non-circular shape is a copy of the cross-sectional shape of a native valve of a subject. In some instances, optionally the native valve of the subject is a bicuspid aortic valve.

The non-circular shape can be imparted on a supportive frame by way of subjecting the frame to a deformation process, which can include a single or multiple deforming steps. It is generally sought to avoid damaging the structural integrity of the supportive frame during such deforming process(es). The undeformed supportive frame may be formed using art known techniques, as described above, or may be obtained from commercial sources and subsequently deformed, as needed, during step (i). In some instances, a deformed supportive frame having a desired non-circular shaped cross-section can be fabricated directly and step (i) can be excluded from the first method, where the already deformed supportive frame is used directly in step (ii).

The non-circular shape of the at least one cross-section of the deformed frame will be maintained even if the supportive frame is crimped, collapsed, or compacted and then re-expanded. For the first method, the deforming process of step (i) can include a temporal exposure to elevated temperature (e.g., about 450° C. to 550° C., or, in some instances, about 520° C.). Such a deforming step can also be considered a shape-setting of the supportive frame. In such instances, the supportive frame (i.e., stent) can be deformed around a mandrel or another structure such that the joints or nodes of the supportive frame are strained into one or more new position(s) and an outer shell can be used to compress the frame into a desired non-circular shaped cross-section along the long axis of the frame. For example, nitinol wire forming a supportive frame can be heated to a suitable temperature and either returned to room temperature or quenched in water to obtain different material properties, including a new shape, which sets the desired non-circular shape in the supportive frame and eliminates the internal stresses of the supportive frame.

### 2. Polymeric Sleeve

In some instances of the methods, the thickness of the embedding polymeric matrix forming the polymeric sleeve can have any suitable thickness thereon. The thickness of the extraluminal polymeric matrix material covering the supportive frame can, for example, when formed by compression molding or other molding techniques have a thickness in a range from about 0 to 500 microns in the circumferential direction and/or a thickness ranging from about 0 to 400 microns in the radial direction of the struts or wire-like elements of the supportive frame. In some instances, an average thickness of about 400 microns in the circumferential direction (i.e., about 200 microns are on each side of the strut) is present. Suitable materials, dimensions, and characteristics of the polymeric sleeve which can be formed according to the methods herein are described above. As previously noted, in some instances, the outer surface may be coated with a gel paving material subsequent to formation of the polymeric sleeve and removal from the mold.

### 3. Polymeric Leaflets

As noted above, the polymeric leaflets of the system can be achieved, for example, by forming the polymeric leaflets and polymeric sleeve (onto the supportive frame) at the same time during the molding process (i.e., single manufacturing step) whereby they form a single polymeric component containing both the polymeric leaflets and polymeric sleeve. The polymeric leaflets can be formed of polymers as previously specified, and are typically made of the same polymer as the polymeric sleeve.

However, in less preferred instances of the methods, polymeric leaflets may be formed/molded as a first component and then the leaflets and a supportive frame can be dip coated into a suitable polymer melt or solution to form the polymeric sleeve. During such instances, the polymer melt or solution forms the polymeric sleeve over the supportive frame and simultaneously attaches to the polymeric leaflets in the mold. Thus, the leaflets become an inseparable part of the formed polymeric sleeve.

The polymeric leaflets forming the valve can have any suitable dimension, shape, or size needed. In most instances, the valve system includes polymeric leaflets which each have a curvilinear or wavy profile in a circumferential axis and are preferably formed/molded in a semi-open conformation, but other conformations are also possible.

In some instances of the first method, the artificial polymeric valve systems include a supportive frame having a non-circular shaped cross-section and the plurality of polymeric leaflets are all symmetrical with respect to the other polymeric leaflets present in the plurality (see FIGS. 2B and 3B). In some other instances of the first method, the artificial polymeric valve systems include a supportive frame having a non-circular shaped cross-section and at least one or more of the plurality of polymeric leaflets is asymmetric with respect to the other polymeric leaflets present in the plurality (see FIGS. 2C and 3C). In still other instances, each of the polymeric leaflets is asymmetrical to all of the other polymeric leaflets in the plurality. In yet other instances, some subset(s) (such as a pair(s) of leaflets) of the plurality of polymeric leaflets may be symmetrical to each other but these subset(s) are not symmetrical to other subset(s) present. It is understood that producing any variation of plurality of polymeric leaflets, having symmetrical, asymmetrical, or combinations of symmetries, can be achieved by the design of the plurality of polymeric leaflets formed in the mold which is used to cast the leaflets.

For the methods described, the polymeric leaflets can each independently have uniform or non-uniform thicknesses. However, in some preferred instances, the polymeric leaflets each independently have a non-uniform (i.e., variable) thickness across any cross-sectional direction of the leaflet. Such variable thickness can be imparted by the selection of the design of the top and bottom cores of the mold in order to produce polymeric leaflets of any desired thickness and having any desired variability in thickness therein, when these mold components are created/fabricated.

### 4. Anti-Leak Flap

The artificial polymeric valve systems formed according to the methods above can also include one or more anti-leak flaps. The anti-leak flaps are defined by the mold which can be designed to produce such flaps on the polymeric sleeve. The anti-leak flaps can be formed of polymers as previously specified, and are typically made of the same polymer as the polymeric sleeve and/or polymeric leaflets. In some instances, the one or more anti-leak flaps include a plurality of optional slits which can improve flexibility and the ability of the anti-leak flap to cover or abut against the native leaflets of a defective valve.

### IV. Uses of Artificial Polymeric Valve Systems

The artificial polymeric valve systems described herein can be used to replace organ valves in an animal subject, such as human. Such organ valves can be diseased, defective, or otherwise compromised valves. Alternatively, the polymeric valve system may be a polymeric heart valve system that is utilized to replace other heart valves; or serve as an extracardiac valve, e.g. in the aorta or other arteries. In still other instances, the polymeric valve system is not limited to use only in the heart or structures therein and may be used in veins; or other luminal structures organs, or organ components of the body of a subject.

The disclosed artificial polymeric valve systems are expected to have a lower occurrence of wear and tear durability failures compared to tissue-based TAVR devices. The disclosed artificial polymeric valve systems are expected to have a lower occurrence of calcific growth within the TAVR polymeric leaflets compared to tissue-based TAVR devices. In some instances, the heart valve systems described herein have a reduction in calcification within the TAVR polymeric leaflets of at least about 10%, 20%, 30%, 40%, 50%, 60%, 70%, 80%, 90%, 95%, 99%, or greater, compared to the native valve. The heart valve system's calcification susceptibility can be determined using a suitable protocol, such as for example, using an in vitro protocol (Boloori, Z. P., et al., Mater Sci Eng C Mater Biol Appi 35: 335-340, 2014) utilizing an accelerated wear testing (AWT) 50 setup with a pro-calcific/phosphorus compound (Golomb G, et al., Biomaterials 12: 397-405, 1991) for at least fifty million valve operation cycles. See also Rotman, O. M., et al., ASAIO J, 66(2), pp. 190-198 for a detailed discussion on durability and stability testing of polymeric transcatheter valves.

In one non-limiting example, the artificial polymeric valve systems described can be used to replace a bicuspid aortic valve (BAV), the most common congenital heart malformation, characterized by the presence of only two valve leaflets with asymmetrical geometry, resulting in elliptical systolic opening. The pathological BAV anatomy often leads to complications stemming from mismatched anatomical features when treated with current valve replacement devices. To mitigate and address these complications, the artificial polymeric valve systems described can be used in BAV patients according to the methods below and the Examples discussed below.

In some instances, the artificial polymeric valve systems can be implanted in a subject in need thereof to replace a defective valve. A non-limiting exemplary method can include the steps of:
(a) inserting an artificial polymeric valve system described herein in a crimped, collapsed, or compacted state into the subject in need thereof;
(b) delivering the artificial polymeric valve system to the defective valve in the subject in need thereof;
(c) implanting the artificial polymeric valve system by expanding the crimped, collapsed, or compacted artificial polymeric valve system into an expanded state, which localizes or fixes the artificial polymeric valve system at the defective valve and replaces the function of the defective valve.

In some instances of the method described above, the defective valve is a vascular or pulmonary valve. In some cases, the vascular valve is a venous valve or a cardiac valve; where the cardiac valve can be selected from an aortic valve, mitral valve, bicuspid aortic valve, tricuspid aortic valve, or a pulmonary valve. In some instances of the above method, the subject is a human and the defective valve is a defective aortic valve.

In some other instances, the valve system may be used in a method of treating a subject in need thereof, the method including the steps of:
(a′) inserting an artificial polymeric valve system of any one of claims 1-23 in a crimped, collapsed, or compacted state into the subject in need thereof;
(b′) delivering the artificial polymeric valve system to an artery, vein, or luminal structure organ in the subject in need thereof;
(c′) implanting the polymeric valve system by expanding the crimped, collapsed, or compacted artificial polymeric valve system into an expanded state, which localizes or fixes the artificial polymeric valve system at the artery, vein, or luminal structure organ.

In some instances of the above methods, the insertion in steps (a) or (a′) involve the use of a delivery catheter or similar extendable member or delivery system. In some cases, the artificial polymeric valve system is crimped, collapsed, or compacted immediately prior to use and then inserted into a delivery catheter. In its crimped, collapsed, or compacted state, the valve system will have either a circular or non-circular shaped cross-section, depending upon the valve system configuration, the patient needs, or application for which it is selected.

Expanding of the crimped, collapsed, or compacted artificial polymeric valve system in steps (c) or (c′) can by performed by balloon expansion, heat expansion, or by relying on inherent superelastic properties of the supportive frame of the system. For instance, in balloon expansion balloon pressure causes plastic deformation of the supportive frame and can also push native (calcific) leaflets of the defective valve away. Regarding heat expansion, the supportive frame can be made, for example, from nitinol and crimped at cold temperatures and plastically deformed into a crimped, collapsed, or compacted profile. Once inside the body, warming to body temperature will cause thermal superelastic behavior, loosening the plastic deformation and gaining the superelastic radial force at body temperature. Supportive frames made of nitinol have superelastic material behavior meaning they exhibit elastic deformation over a large range of strains without plastically deforming. In some instances, this represents the primary expansion method of the crimped, collapsed, or compacted artificial polymeric valve system in steps (c) or (c′) of the above methods.

In some instances of the above methods, during steps (b)/(b′) or (c)/(c′), the artificial polymeric valve system in the crimped, collapsed, or compacted state is rotated and/or twisted, prior to expansion, to align a long axis of the defective valve with the long axis of the artificial polymeric valve system. For instance, the delivery catheter can be used to align, by rotation and/or twisting, the aforementioned axes before the polymeric valve system is expanded during steps (c) or (c′) of the above methods. This allows for alignment of the leaflets and the supportive frame (i.e., stent) for improved function. In some instances, the alignment step can be accomplished with a rotational component of a delivery handle that is directly attached to the delivery catheter body. The alignment would be confirmed prior to deployment of the valve system using, for example, radiopaque markers on the supportive frame (i.e., stent) and angiographic assessment of the native defective valve, such as aortic valve. Adjusting the rotational component (such as by rotating it from about −60° to about +60°, or any subranges therein) to align with the geometry of native valve during the deployment of the system.

The disclosed artificial polymeric valve systems and methods can be further understood through the following numbered paragraphs.

1. An artificial polymeric valve system comprising:
a supportive frame and a plurality of polymeric leaflets;
wherein the supportive frame comprises a plurality of openings, wherein the supportive frame has a long axis and at least one or more cross-sections taken orthogonally to the long axis have a non-circular shape, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end; and
wherein the polymeric leaflets are able to open and close at an operative end, wherein when the plurality of polymeric leaflets is in the closed position, the operative ends of the leaflets abut each other.

2. The system of paragraph 1, wherein the non-circular shape is selected from elliptical, lemniscate, cardioid, quartic bean curve, or polygonal; or the non-circular shape comprises fluctuating asymmetry; or the non-circular shape is a copy of the cross-sectional shape of a native valve of a subject, and optionally the native valve of the subject is a bicuspid aortic valve.

3. An artificial polymeric valve system comprising:
a supportive frame and a plurality of polymeric leaflets;
wherein the supportive frame comprises a plurality of openings, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end;
wherein at least one or more of the plurality of polymeric leaflets is asymmetrical to the other polymeric leaflets in the plurality; and
wherein the polymeric leaflets are able to open and close at an operative end, wherein when the plurality of polymeric leaflets is in the closed position, the operative ends of the leaflets abut each other.

4. The system of any one of paragraphs 1-3, wherein the number of polymeric leaflets forming the plurality of polymeric leaflets is selected from two, three, four, five, six, seven, eight, nine, ten, eleven, or twelve polymeric leaflets.

5. The system of any one of paragraphs 1-2, wherein each of the polymeric leaflets is symmetrical to the other polymeric leaflets in the plurality.

6. The system of any one of paragraphs 1-2 and 4, wherein at least one or more of the polymeric leaflets is asymmetrical to the other polymeric leaflets in the plurality.

7. The system of any one of paragraphs 1-4, wherein each of the polymeric leaflets is asymmetrical with the other polymeric leaflets in the plurality.

8. The system of any one of paragraphs 1-7, wherein the supportive frame is a continuous material or is formed from a plurality of connected struts, wherein each strut is connected to one or more other struts in the plurality via a joint or node.

9. The system of paragraph 8, wherein each of the plurality of connected struts comprises an anchor and/or wherein each of the plurality of the connected struts and/or the one or more joints each comprises indentations and/or openings.

10. The system of paragraph 8, wherein the connected struts comprise one or more surfaces having surface features ranging from smooth and featureless to others having micro and/or macro asperities and the one or more surfaces are not exposed in order to prevent risk of thrombus formation.

11. The system of any one of paragraphs 1-10, wherein the supportive frame further comprises a crown region in which the polymeric matrix is not located,
optionally wherein the crown region comprises one or more anchors.

12. The system of any one of paragraphs 1-11, wherein the supportive frame is in the form of an armature or scaffold-like construct which is expandable.

13. The system of any one of paragraphs 1-12, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.

14. The system of any one of paragraphs 1-13, wherein the polymeric sleeve is made of a biocompatible, hemocompatible, and/or mechanically stable polymer.

15. The system of any one of paragraphs 1-13, wherein the polymeric sleeve comprises one or more polymers selected from the group consisting of thermoplastic polymers/elastomers, thermoset polymers, thermally crosslinkable polymers, elastomeric polymer biomaterials, polymers containing polysulfone(s), PTFE, crosslinked poly(styrene-isobutylenese-styrene) (xSIBS), poly(styrene-isobutylene-styrene) (SIBS), polymyrcene, polymenthide, and poly(F-decalactone), silicones, polyolefins, polydiene elastomers, poly(vinyl chloride), natural rubbers, heparinized polymers, hydrogels, polypeptides elastomers, polysiloxane-urea elastomers, and polyurethanes.

16. The system of any one of paragraphs 1-15, wherein the polymeric sleeve further comprises one or more anti-leak flaps thereon.

17. The system of any one of paragraphs 1-16, wherein the polymeric sleeve further comprises a gel paving material on one or more outer surfaces of the polymeric sleeve.

18. The system of any one of paragraphs 1-17, wherein the polymeric sleeve has a thickness of about 0 to 500 μm or sub-ranges therein.

19. The system of any one of paragraphs 1-18, wherein each of the polymeric leaflets in the plurality comprises variable thickness having a center line of symmetry, wherein a given cross-section each of the leaflets has at least two or more thicknesses.

20. The system of paragraph 19, wherein each of the leaflets has a minimum thickness in a range from about 50 to 200 μm

21. The system of paragraph 19, wherein each of the leaflets has a maximum thickness in a range from about 200 to 600 μm.

22. The system of any one of paragraphs 1-21, wherein the polymeric sleeve is formed from the same polymeric material as the polymeric leaflets, or from a different polymeric material than the polymeric leaflets.

23. The system of any one of paragraphs 1-22, wherein the supportive frame and/or supportive sleeve comprise radiopaque materials.

24. A method of making an artificial polymeric valve system, the method comprising the steps of:
(i) deforming a supportive frame, wherein the supportive frame has a long axis and at least one or more cross-sections taken orthogonally to the long axis have a non-circular shape after the supportive frame is deformed;
(ii) placing the deformed supportive frame inside a cavity of a mold in a position suitable to facilitate flow of the polymer around the deformed supportive frame,
wherein the mold comprises a top core, a bottom core, and one or more encasement components which encase the top and bottom cores,
wherein the top core and the bottom core define a shape to form a plurality of polymeric leaflets in a suitable orientation wherein the plurality of polymeric leaflets are formed optionally in a semi-open position having a zero residual stress conformation,
optionally, wherein the shape forming the plurality of polymeric leaflets has a curvilinear profile along a circumferential axis of the polymeric leaflets being formed; and
(iii) introducing at least one polymer into the mold;
(iv) molding or casting the plurality of polymeric leaflets and a polymeric sleeve around the deformed supportive frame to form the artificial polymeric valve system, wherein the plurality of polymeric leaflets and the polymeric sleeve are a continuum and each of the polymeric leaflets are connected to the polymeric sleeve at an attachment end; and
(v) removing the artificial polymeric valve system formed from the mold.

25. The method of paragraph 24, wherein the non-circular shape is selected from elliptical, lemniscate, cardioid, quartic bean curve, or polygonal; or the non-circular shape comprises fluctuating asymmetry; or the non-circular shape is a copy of the cross-sectional of a shape of a native valve of a subject, and optionally the native valve of the subject is a bicuspid aortic valve.

26. The method of any one of paragraphs 24-25, wherein the number of polymeric leaflets formed in step (iv) is two, three, four, five, six, seven, eight, nine, ten, eleven, or twelve polymeric leaflets.

27. The method of any one of paragraphs 24-26, wherein each of the plurality of polymeric leaflets present are symmetrical to each other.

28. The method of any one of paragraphs 24-26, wherein each of the plurality of polymeric leaflets present are asymmetrical to each other.

29. The method of any one of paragraphs 24-26, wherein at least one of the polymeric leaflets present is not symmetrical with the other polymeric leaflets in the plurality.

30. The method of any one of paragraphs 24-29, wherein the one or more encasement components further comprise one or more arched segments or pins and/or one or more sealing elements;
wherein the one or more arched segments or pins hold the supportive frame.

31. The method of any one of paragraphs 24-30, wherein the method further comprises a mold cleaning step prior to step (i) or (ii).

32. The method of any one of paragraphs 24-31, wherein the mold is heated during steps (iii) and/or (iv) to a temperature ranging from about 100° C. to 350° C.

33. The method of any one of paragraphs 24-32, wherein step (iii) comprises applying pressure in a range from about 0.01 to about 10 tons by way of a plunger to spread the polymer through the mold.

34. The method of any one of paragraphs 24-33, wherein step (iv) is performed by a compression molding process, transfer molding process, or injection molding process.

35. The method of any one of paragraphs 24-33, wherein step (iv) is performed by dip coating process.

36. The method of any one of paragraphs 24-35, further comprising, prior to step (ii), applying one or more release agents to the surfaces of the mold to facilitate removal of the artificial polymeric valve system after formation.

37. The method of any one of paragraphs 24-36, wherein the supportive frame is formed from a plurality of connected struts each connected to one or more other struts via a joint.

38. The method of any one of paragraphs 24-36, wherein the supportive frame is in the form of an armature or scaffold-like construct which is expandable.

39. The method of any one of paragraphs 24-38, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.

40. The method of any one of paragraphs 24-39, wherein the at least one polymer is selected from one or more of the group consisting of thermoplastic polymers/elastomers, thermoset polymers, thermally crosslinkable polymers, elastomeric polymer biomaterials, polymers containing polysulfone(s), PTFE, crosslinked poly(styrene-isobutylenese-styrene) (xSIBS), poly(styrene-isobutylene-styrene) (SIBS), polymyrcene, polymenthide, and poly(F-decalactone), silicones, polyolefins, polydiene elastomers, poly(vinyl chloride), natural rubbers, heparinized polymers, hydrogels, polypeptides elastomers, polysiloxane-urea elastomers, and polyurethanes.

41. The method of any one of paragraphs 24-40, wherein the polymeric sleeve further comprises one or more anti-leak flaps thereon.

42. The method of any one of paragraphs 24-41, wherein the method further comprises a step of coating the polymeric sleeve, after step (v) with a gel paving material onto one or more outer surfaces of the polymeric sleeve.

43. The method of any one of paragraphs 24-42, wherein each of the plurality of polymeric leaflets comprises variable thickness across cross-sections of each of the polymeric leaflets running from an attachment end to an operative end thereof.

44. The method paragraph 43, wherein each of the plurality of polymeric leaflets has a minimum thickness in a range from about 50 to 200 μm.

45. The method of any one of paragraphs 43 or 44, wherein each of the plurality of polymeric leaflets has a maximum thickness in a range from about 200 to 600 μm.

46. The method of any one of paragraphs 24-45, wherein step (i) comprises placing the supportive frame inside the cavity of the mold while inducing the supportive frame to comprise a degree of oversizing during molding or casting step (iv).

47. A method of making an artificial polymeric valve system, the method comprising the steps of:
(i′) placing a supportive frame inside a cavity of a mold in a position suitable to facilitate flow of the polymer around the supportive frame,
wherein the mold comprises a top core, a bottom core, and one or more encasement components which encase the top and bottom cores,
wherein the top core and the bottom core define a shape to form a plurality of polymeric leaflets in a suitable orientation wherein the plurality of polymeric leaflets are formed preferably in a semi-open position having a zero residual stress conformation,
optionally, wherein the shape forming the polymeric leaflets has a curvilinear profile along a circumferential axis of the polymeric leaflets being formed; and
(ii′) introducing at least one polymer into the mold;
(iii′) molding or casting the plurality of polymeric leaflets and a polymeric sleeve around the supportive frame to form an artificial polymeric valve system,
wherein at least one or more of the plurality of polymeric leaflets formed is asymmetrical to the other polymeric leaflets in the plurality;
wherein the plurality of polymeric leaflets and the polymeric sleeve are a continuum and each of the polymeric leaflets are connected to the polymeric sleeve at an attachment end; and
(iv′) removing the artificial polymeric valve system formed from the mold.

48. The method of paragraph 47, wherein the number of polymeric leaflets formed in step (iii′) is two, three, four, five, six, seven, eight, nine, ten, eleven, or twelve polymeric leaflets.

49. The method of any one of paragraphs 47-48, wherein each of the plurality of polymeric leaflets present are asymmetrical to each other.

50. The method of any one of paragraphs 47-49, wherein the one or more encasement components further comprise one or more arched segments or pins and/or one or more sealing elements;
wherein the one or more arched segments or pins hold the supportive frame.

51. The method of any one of paragraphs 47-50, wherein the method further comprises a mold cleaning step prior to step (i′).

52. The method of any one of paragraphs 47-51, wherein the mold is heated during steps (ii′) and/or (iii′) to a temperature ranging from about 100° C. to 350° C.

53. The method of any one of paragraphs 47-52, wherein step (iii) comprises applying pressure in a range from about 0.01 to about 10 tons by way of a plunger to spread the polymer through the mold.

54. The method of any one of paragraphs 47-53, wherein step (iii′) is performed by a compression molding process, transfer molding process, or injection molding process.

55. The method of any one of paragraphs 47-53, wherein step (iii′) is performed by dip coating process.

56. The method of any one of paragraphs 47-55, further comprising, prior to step (i′), applying one or more release agents to the surfaces of the mold to facilitate removal of the artificial polymeric valve system after formation.

57. The method of any one of paragraphs 47-56, wherein the supportive frame is formed from a plurality of connected struts each connected to one or more other struts via a joint.

58. The method of any one of paragraphs 47-56, wherein the supportive frame is in the form of an armature or scaffold-like construct which is expandable.

59. The method of any one of paragraphs 47-58, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.

60. The method of any one of paragraphs 47-59, wherein the at least one polymer is selected from one or more of the group consisting of thermoplastic polymers/elastomers, thermoset polymers, thermally crosslinkable polymers, elastomeric polymer biomaterials, polymers containing polysulfone(s), PTFE, crosslinked poly(styrene-isobutylenese-styrene) (xSIBS), poly(styrene-isobutylene-styrene) (SIBS), polymyrcene, polymenthide, and poly(ε-decalactone), silicones, polyolefins, polydiene elastomers, poly(vinyl chloride), natural rubbers, heparinized polymers, hydrogels, polypeptides elastomers, polysiloxane-urea elastomers, and polyurethanes.

61. The method of any one of paragraphs 47-60, wherein the polymeric sleeve further comprises one or more anti-leak flaps thereon.

62. The method of any one of paragraphs 47-61, wherein the method further comprises a step of coating the polymeric sleeve, after step (iv′) with a gel paving material onto one or more outer surfaces of the polymeric sleeve.

63. The method of any one of paragraphs 47-62, wherein each of the plurality of polymeric leaflets comprises variable thickness across cross-sections of each of the polymeric leaflets running from an attachment end to an operative end thereof.

64. The method paragraph 63, wherein each of the plurality of polymeric leaflets has a minimum thickness in a range from about 50 to 200 μm.

65. The method of any one of paragraphs 63 or 64, wherein each of the plurality of polymeric leaflets has a maximum thickness in a range from about 200 to 600 μm.

66. The method of any one of paragraphs 47-65, wherein step (i) comprises placing the supportive frame inside the cavity of the mold while inducing the supportive frame to comprise a degree of oversizing during molding or casting step (iii′).

67. An artificial polymeric valve system formed according to the method of any one of paragraphs 24-66.

68. A method of replacing a defective valve in a subject in need thereof, the method comprising the steps of:
(a) inserting an artificial polymeric valve system of any one of paragraphs 1-23 in a crimped, collapsed, or compacted state into the subject in need thereof;
(b) delivering the artificial polymeric valve system to the defective valve in the subject in need thereof;
(c) implanting the artificial polymeric valve system by expanding the crimped, collapsed, or compacted artificial polymeric valve system into an expanded state, which localizes or fixes the artificial polymeric valve system at the defective valve and replaces the function of the defective valve.

69. The method of paragraph 68, wherein the defective valve is vascular valve, pulmonary valve, or urinary valve.

70. The method of paragraph 69, wherein the vascular valve is a venous valve or a cardiac valve; optionally wherein the cardiac valve is selected from the group consisting of aortic valve, mitral valve, bicuspid aortic valve, tricuspid aortic valve, or pulmonary valve.

71. The method of any one of paragraphs 68-70, wherein the artificial polymeric valve system in the crimped, collapsed, or compacted state is inserted during step (a) via a delivery catheter.

72. The method of any one of paragraphs 68-71, wherein the artificial polymeric valve system is crimped, collapsed, or compacted state prior to step (a).

73. The method of any one of paragraphs 68-72, wherein expanding of the crimped, collapsed, or compacted artificial polymeric valve system in step (c) is performed by balloon expansion, heat expansion, or inherent superelastic properties.

74. The method of any one of paragraphs 68-73, wherein during steps (b) or (c) the artificial polymeric valve system in the crimped, collapsed, or compacted state is rotated and/or twisted, prior to expansion, to align a long axis of the defective valve with the long axis of the artificial polymeric valve system.

75. A method of treating a subject in need thereof, the method comprising the steps of:
(a′) inserting an artificial polymeric valve system of any one of paragraphs 1-23 in a crimped, collapsed, or compacted state into the subject in need thereof;
(b′) delivering the artificial polymeric valve system to an artery, vein, or luminal structure organ in the subject in need thereof;
(c′) implanting the polymeric valve system by expanding the crimped, collapsed, or compacted artificial polymeric valve system into an expanded state, which localizes or fixes the artificial polymeric valve system at the artery, vein, or luminal structure organ.

### Examples

### Materials and Methods:

### Asymmetric Leaflet Implants:

Asymmetric polymeric leaflet implants were designed parametrically using SolidWorks (DS SolidWorks Corp., Waltham, MA).

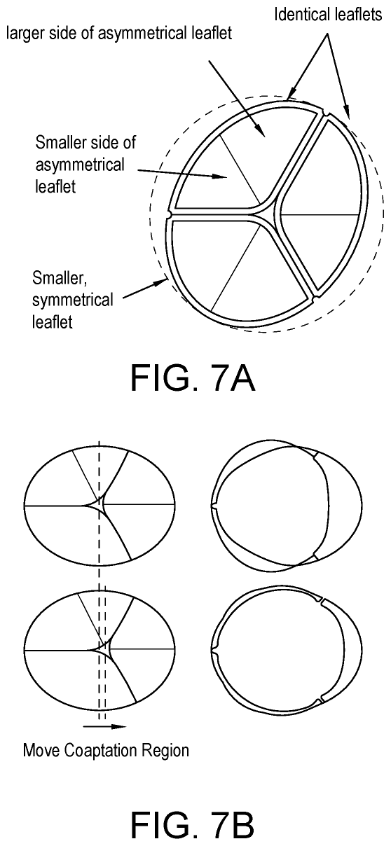

As shown in FIG. 7A, an initial polymeric leaflet implant with an ellipticity index of 1.25 (major axis divided by minor axis), derived from the average ellipticity of the annulus of the patient-specific models was used. The major and minor diameters were chosen such that the projected area of the implant was approximately equal to that of a commercial valve (Evolut R 29 mm, Medtronic plc, Dublin, Ireland). The polymeric leaflet implant shown in FIG. 7A incorporates two identical, asymmetrical polymeric leaflets, and a smaller, symmetrical polymeric leaflet. This implant was tested in silico in Abaqus 2021 (SIMULIA, Dassault Systemes, Providence, RI) using an explicit, dynamic analysis, where a pressure gradient waveform boundary condition (with an average systolic and diastolic pressure gradient of 10 mmHg and 61 mmHg, respectively, at 60 BPM) was applied to the polymeric leaflets' surface to observe their motion throughout a cardiac cycle. The polymeric leaflets were meshed with 89,076 C3D8R elements, frictional hard contact (0.1 friction coefficient) was defined between the ventricular side of each polymeric leaflet, and mass scaling was employed to prevent time step size from falling below 1×10⁻⁷.

The polymeric leaflet implant was designed using the polymer, xSIBS, material properties—a highly durable novel thermoset polymer, which has shown excellent performance in previous TAVR studies (Kovarovic B, et al., Journal of biomechanical engineering. 2022; 144(6); Rotman O M, et al., American Society for Artificial Internal Organs: 1992). 2020; 66(2):190-8; Rotman O M, et al., Annals of biomedical engineering. 2019; 47(1):113-25). An Arruda-Boyce material model was used to represent the hyperelastic behavior of the xSIBS polymer (μ=1.2402, λₘ=3.51291, D=0). Polymeric leaflet stress analysis was presented with von Mises stresses given the assumed isotropic material properties of the xSIBS (Kovarovic B, et al., Journal of biomechanical engineering. 2022; 144(6)); however, principal stresses were additionally analyzed.

Design deficiencies identified in the initial cardiac cycle simulations were mitigated by adjusting the leaflet dimensions in SolidWorks. The coaptation region, or surface area of polymeric leaflet abutment towards the adjoining aortic wall, and the free edge of the initial design (see FIG. 7B leftmost dashed line) were adjusted iteratively in order to obtain an improved design (see FIG. 7B rightmost dashed line) which demonstrated a 16% increased geometric orifice area (GOA) and a 25% reduced peak commissural stress. Further optimization was performed on the polymeric leaflet thickness following the methodology described in previous studies (Kovarovic B, et al., Journal of biomechanical engineering. 2022; 144(6)); which was applied to the asymmetric leaflets design. Briefly, a MATLAB code was used to modify the polymeric leaflet thickness to improve its overall stress distribution, resulting in a decrease in the peak stress near the attachment region by 20% and additional decrease in stresses in the belly of the leaflet. After these improvements, the leaflets' motion and stresses were optimized.

### Selection of Commercial Stent and Device Modeling:

An Evolut self-expandable stent was used for both the asymmetric valve and commercial Evolut R 29 mm valve to observe the impact of only the polymeric leaflet design. The commercial model, utilized in previous studies (Anam S B, et al., J Cardiovasc Transl Res. 2021, 10.1007/s12265-021-10191-z; Anam S B, et al., Cardiovasc Eng Technol. 2022, 10.1007/s13239-022-00620-8; Bianchi M, et al., Biomechanics and modeling in mechanobiology. 2019; 18(2):435-51; Ghosh R P, et al., Biomechanics and modeling in mechanobiology. 2020, 10.1007/s10237-020-01304-9), has superelastic nitinol material properties which were validated in Abaqus/Standard using in vitro radial force data (Egron S, et al., ASAIO Journal. 2018; 64(4):536-43) to ensure accurate deployments. Its corresponding leaflets were assigned a hyperelastic material property for glutaraldehyde-treated porcine pericardial tissue (Caballero A, et al., J Mech Behav Biomed Mater. 2017; 75:486-94) and were meshed with C3D8R elements with a similar density as the asymmetric leaflets.

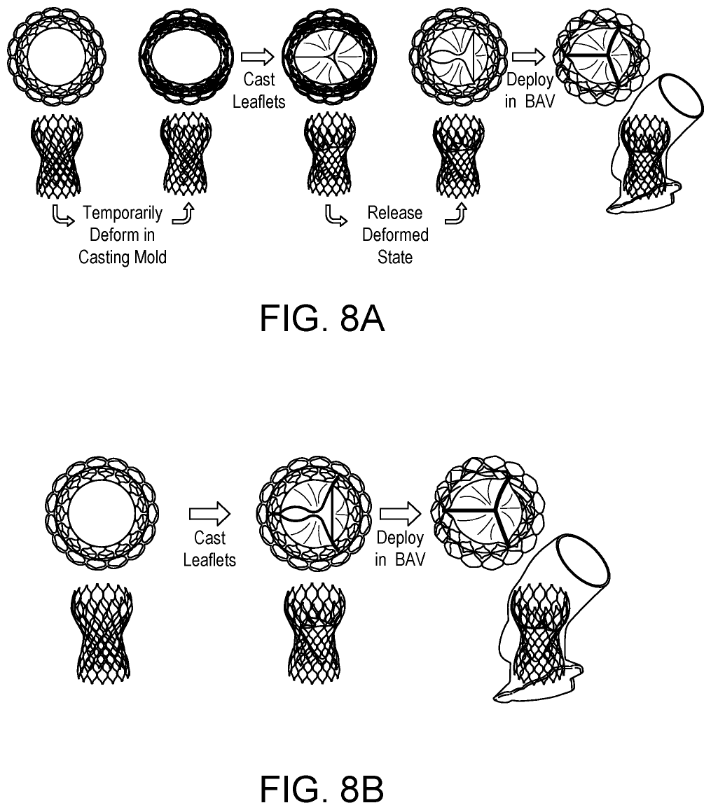

To incorporate the asymmetrical polymeric leaflets into the commercial stent frame, they were attached via the following in silico implementation process (which was modeled and performed only in silico with finite element analysis (FEA)) (see FIG. 8A).

Overall, a circular stent is compressed into a casting mold that temporarily (and elastically) deforms it into the eccentric valve shape (in silico: an eccentric mandrel shape-set the circular waist stent into an elliptical waist stent) (see FIG. 8A (leftmost)). The polymeric asymmetric leaflets are then cast into this mold (in silico: polymeric leaflets were numerically tied to the stent frame), and the stent is released from its deformed state returning to its original circular cross-section (in silico: mandrel shape-setting boundary conditions are reversed) (see FIG. 8A (center)).

Although the polymeric leaflets are now residing in a stressed state, the stress magnitudes are minimal, and considering that xSIBS presents little to no structural damage even after eight days in a fully crimped state, this is negligible.

Upon deployment of the valve in a bicuspid aortic valve (BAV) patient (in silico: patient-specific deployment operation), the stent assumed an elliptical shape commonly seen in BAV anatomies, and the polymeric leaflets experience stresses that are close to their original stress-free state (see FIG. 8A (rightmost)). The polymeric artificial valve design combined with the simulated implementation technique resulted in asymmetrical polymeric leaflets within an elliptical orifice. This is in contrast to the current TAVR valves having symmetric polymeric leaflets that are deployed in an elliptical orifice.

### Patient-Specific Models and Material Selection:

The patient-specific models used in this Example were reconstructed from six type 1 bicuspid aortic valve (BAV) patients that received a 29 mm self-expandable TAVR (not shown).

De-identified preoperative CT scans of these patients were obtained from Rabin Medical Center (Petah Tikva, Israel) under Stony Brook IRB approved protocol (522113). ScanIP (Synopsys, Mountain View, CA) was used to segment out the relevant anatomy using procedures outlined in a previous study (Bianchi M, et al., Artificial organs. 2016; 40(12): E292-e304). An Ogden third-order model was used to fit hyperelastic material data for the aortic wall tissue (Martin C, et al., Eur J Cardiothorac Surg. 2011; 40(1):28-34) and the leaflets (Martin C, et al., J Biomed Mater Res A. 2012; 100(6):1591-9), where the fused leaflet received an average of the two individual leaflet properties (Anam S B, et al., J Cardiovasc Transl Res. 2021, 10.1007/s12265-021-10191-z; Emendi M, et al., Annals of biomedical engineering. 2021; 49(2):627-41). A linear elastic material model was used for the calcium deposits (E=12.5 MPa, v=0.3) (Bianchi M, et al., Artificial organs. 2016; 40(12):E292-e304). All aortic root components and stent models were meshed finely with C3D4 and C3D8R elements, respectively.

### Finite Element Analysis of Transcatheter Aortic Valve Deployment:

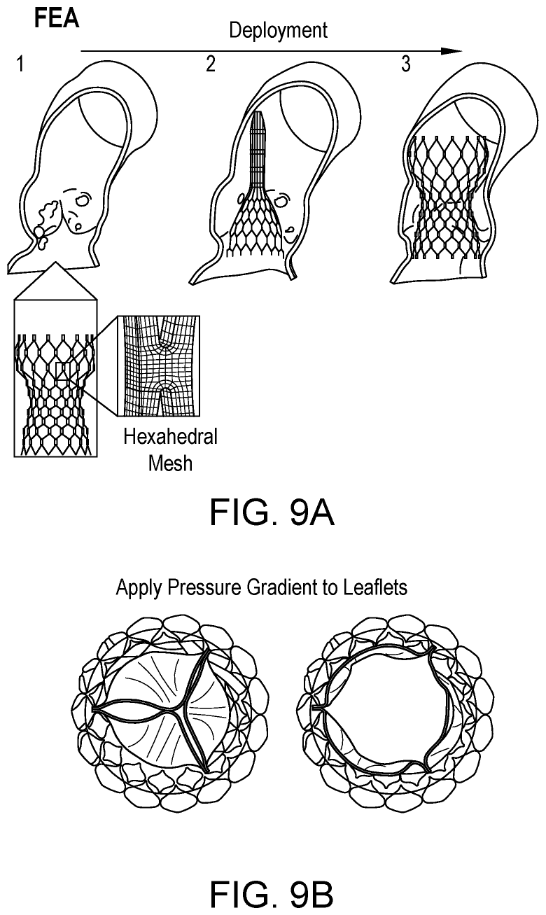

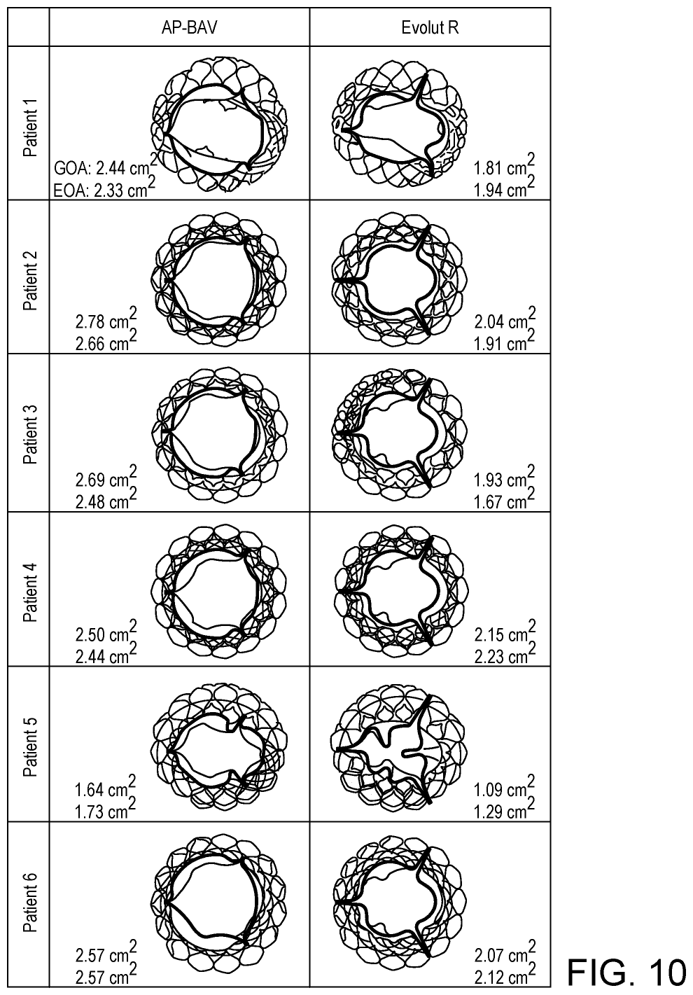

Finite element analysis (FEA) simulations of the transcatheter aortic valve replacement (TAVR) procedure were performed following similar methods described in previous studies (Anam S B, et al., J Cardiovasc Transl Res. 2021, 10.1007/s12265-021-10191-z; Anam S B, et al., Cardiovasc Eng Technol. 2022, 10.1007/s13239-022-00620-8; Ghosh R, et al., Journal of biomechanical engineering. 2018, 10.1115/1.4040600). Briefly, the stent was crimped and deployed 5 mm below the aortic annulus following manufacturer's guidelines (Bianchi M, et al., Biomechanics and modeling in mechanobiology. 2019; 18(2):435-51)²⁴ (see FIG. 9A; Deployment 1-2-3). The polymeric leaflets were added to the stent afterward using the displacement field of the stent during deployment to map the leaflets to their deformed configuration (see FIG. 9B leftmost). The asymmetric polymeric leaflets were connected to the stent in a consistent orientation such that its long axis aligned with the axis passing through the two BAV commissures. Similarly, one of the commissures in the Evolut R leaflets was also oriented along this axis through the BAV commissures. In their post-deployment state, a pressure gradient was applied to the aortic surface of the leaflets to simulate a cardiac cycle, and the peak systolic configuration (see FIG. 9B rightmost) was extracted for GOA calculation and for the computational fluid dynamics (CFD) simulations discussed below.

### Computational Fluid Dynamics (CFD) Setup:

The deployed stent, peak systolic polymeric leaflets, and BAV anatomy were all transferred to ANSYS Fluent (ANSYS, Inc., Canonsburg, PA) for CFD meshing and transient fixed geometry CFD simulations. Laminar flow was assumed with Newtonian properties with viscosity of 0.0035 kg m⁻¹ s⁻¹ and density of 1100 kg m⁻³ to simulate the blood flow.

Two different simulations were created for the hemodynamic analysis, and third cycle results were only considered. First, to record the volume flow rate (VFR) through each valve and calculate their effective orifice area (EOA), (Ghosh R P, et al., Biomechanics and modeling in mechanobiology. 2020, 10.1007/s10237-020-01304-9) a fixed pressure gradient defining a cardiac cycle was applied to the inlet and zero pressure to the outlet.

The second set of simulations used mass flow rate (MFR) as the inlet boundary condition (with zero pressure at the outlet). The MFR was calculated on a per patient basis according to the VFR from the eccentric valve model recorded in the first set of simulations. Thus, the two polymeric leaflet models for the same patient received the same boundary conditions, but the MFR differed between patients to maintain each of their physiological flow rate magnitudes. The MFR simulations were used because, according to mass conservation, fixing the flow rate between the models allows for a direct comparison of the jet flow velocity which differs based on the orifice area. The jet flow velocity and the WSS that was imposed on the aortic walls were extracted from the MFR simulations.

### Statistical Analysis:

Using SPSS (SPSS Inc., Chicago, IL), a non-parametric, independent samples Mann-Whitney U test was used to compare the EOA, maximum jet velocity, and maximum WSS values between the asymmetric models and the Evolut R models. A p-value of 0.05 was used to determine significance.

### Results:

For clarifying the comparison between the Evolut R and the polymeric leaflet valve structure developed, the latter will be termed AP-BAV (asymmetric polymeric BAV). FIG. 9A shows the results of the FEA deployments in patient-specific models from the aortic view at peak systole. Visually, the AP-BAV leaflets appear to open more completely, with the free edge positioned close to the stent frame producing unobstructed lumens. The Evolut R leaflets, on the other hand, appear more restricted with their free edges often remaining at a larger distance from the stent frame, thus limiting their orifice area.

Patients 1 and 5, with heavy calcium deposits on both native leaflets, produced the most elliptical orifices with stent eccentricities of 1.66 and 1.32 at the level of the supra-annular leaflets. The other patients, which had less severe calcifications (heavy calcium on only one native leaflet or minor calcium deposits on both native leaflets), had more circular orifices despite the anatomy of the BAV, with eccentricities of 1.04 (patient 2), 1.07 (patient 3), and 1.13 (patients 4 and 6). The highly eccentric deployments in patients 1 and 5 had an adverse effect on the leaflet shape of the Evolut R: in patient 1, the bottom leaflet spans the long axis of the eccentric orifice, causing the center of that leaflet to protrude into the lumen, and in patient 5 the valve experienced folding at the center of all three leaflets resulting in a clover-shaped opening. The asymmetric polymeric BAV leaflets on the other hand, open nearly completely in these two patients producing a mostly unobstructed lumen.

The peak systolic performance is quantitatively compared via the GOA and EOA which are both listed in FIG. 10. For the AP-BAV (median [IQR], GOA: 2.54 cm² [2.44-2.69], EOA: 2.46 cm² [2.33-2.57]), patient 2 had the largest GOA and EOA (2.78 cm² and 2.66 cm², respectively). For the Evolut R (median [IQR], GOA: 1.99 cm² [1.81-2.07], EOA: 1.93 cm² [1.67-2.12]), patient 4 had the largest GOA and EOA (2.15 cm² and 2.23 cm², respectively). The smallest orifice areas appear in patient 5 for both the AP-BAV (GOA=1.64 cm², EOA=1.73 cm²) and Evolut R (GOA=1.09 cm², EOA=1.29 cm²). In all patients, the GOA and EOA are significantly larger for the asymmetric polymeric BAV as compared to the Evolut R valve (p=0.026). The largest discrepancy occurred in patient 3 where the EOA of the AP-BAV exceeds that of the Evolut R by 48% (2.48 cm² vs 1.67 cm²). This was followed by patients 2, 5, 6, 1, and 4 where the AP-BAV produced an EOA 40%, 33%, 21%, 20%, and 10% larger than the Evolut R valve, respectively.

The peak systolic velocity contours were plotted on a cross-section of the CFD domain parallel to the central axis of the stent and also showed the transvalvular jet flow. All jet flows were found to be approximately parallel to the left ventricular outflow track (LVOT), and each began to dissipate as it reached the aortic arch. All models had a uniform velocity contour within the jet flow except for the Evolut R model in patient 5 where a streak of lower velocity appeared in the jet flow due to the leaflet belly protruding into the center of the lumen.

The peak systolic jet flow velocities are listed in Table 1 below, which were the highest velocities that appeared within the jet flow (sampled downstream approximately 1.4 cm below the aortic end of the stent).

TABLE 1
Peak Systolic Jet Flow Velocities

| Patient # | AP-BAV | Evolut R |
|---|---|---|
| Patient 1 | 1.91 m/s | 2.31 m/s |
| Patient 2 | 2.01 m/s | 2.89 m/s |
| Patient 3 | 2.03 m/s | 3.11 m/s |
| Patient 4 | 2.03 m/s | 2.19 m/s |
| Patient 5 | 1.95 m/s | 2.65 m/s |
| Patient 6 | 2.11 m/s | 2.57 m/s |

The two models (AP-BAV and Evolut R) within each patient case have the same MFR boundary conditions. Thus, a comparative study can be performed on a per patient basis. The maximum velocity was significantly higher through the Evolut R (median [IQR], 2.61 m/s [2.31-2.89]) than through the asymmetric polymeric AP-BAV (median [IQR], 2.02 m/s [1.95-2.03]) for all patients (p=0.002). The largest difference in maximum jet velocity was observed in patient 3 where the velocity through the Evolut R was 53% larger than the velocity through the AP-BAV (3.11 m/s vs 2.03 m/s). The remaining patients, ordered from largest to smallest difference in jet velocity, were as follows: patient 2, 5, 6, 1, and 4, where the jet velocity through the Evolut R exceeded the AP-BAV by 44%, 36%, 22%, 21% and 8%, respectively.

Wall shear stress (WSS) magnitudes on the aortic walls were determined for each patient and each leaflet model. The WSS concentrations are mainly located at the base of the ascending aorta, but some patients show elevated WSS levels near the sinuses as well.

The maximum WSS is given in Table 2 below and the pair of models tested for each patient can be compared directly since they received the same MFR boundary condition.

TABLE 2
Maximum Wall Shear Stress (WSS) Magnitudes

| Patient # | AP-BAV | Evolut R | |
|---|---|---|---|
| Patient 1 | 57.9 Pa | 71.6 | Pa |
| Patient 2 | 58.8 Pa | 101.2 | Pa |
| Patient 3 | 65.3 Pa | 104.5 | Pa |
| Patient 4 | 52.9 Pa | 69.7 | Pa |
| Patient 5 | 51.2 Pa | 104.2 | Pa |
| Patient 6 | 66.6 Pa | 84.3 | Pa |

The WSS was found to be significantly higher in the Evolut R models (median [IQR], 92.75 Pa [71.6-104.2]), as compared to the AP-BAV models (median [IQR], 58.35 Pa [52.9-65.3]) in all patients (p=0.002). The largest difference was seen in patient 5 where the Evolut R exceeded the AP-BAV by 103% (104.2 Pa vs 51.2 Pa). However, this maximum value is not located in the same region as in the rest of the models. Instead, it resides in the sinus region. For the rest of the patients that show maximum WSS at the base of the ascending aorta, the ranking from largest to smallest difference in WSS between the valves is as follows: patient 2, 3, 4, 6, and 1, where the WSS in the Evolut R models exceeds the AP-BAV models by 72%, 60%, 32%, 27%, and 24%, respectively.

### Discussion:

In the study discussed herein, an asymmetric polymeric TAVR valve designed for bicuspid aortic valve (BAV) patients was developed and analyzed.

When designing and testing the asymmetrical leaflets in silico, the goal was to keep stresses below 2 MPa—the fatigue limit of the xSIBS material (Kovarovic B, et al., Journal of biomechanical engineering. 2022; 144(6)). After design improvements and thickness optimization, the maximum von Mises stress was 0.81 MPa, far below the fatigue limit as well as the xSIBS yield strength (5 MPa). The leaflets of the tricuspid TAVR valve, PolyV1 (developed by Polynova Cardiovascular Inc.), that are made from the same material and similarly optimized, have exceeded 900 million cycles of in vitro durability testing while maintaining excellent hemodynamic performance (Kovarovic B, et al., Journal of biomechanical engineering. 2022; 144(6); Rotman O M, et al., American Society for Artificial Internal Organs: 1992). 2020; 66(2):190-8; Rotman O M, et al., Annals of biomedical engineering. 2019; 47(1):113-25). As the asymmetric polymeric leaflets share similar stress magnitudes, it is reasonable to assume that similar long-term durability can be achieved.

An advantage of using xSIBS for the asymmetric polymeric leaflets is its biocompatibility, resistance to calcification, and ease of manufacturing. The xSIBS PolyV1 polymeric leaflets showed significantly less platelet activation and calcium deposition than tissue valves, (Kovarovic B, et al., Journal of biomechanical engineering. 2022; 144(6); Rotman O M, et al., American Society for Artificial Internal Organs: 1992). 2020; 66(2):190-8) which often develop calcifications over time, leading to degeneration of performance and durability. Additionally, because the polymeric leaflets are manufactured by molding, the production time and cost are greatly reduced, and the artificial valve can be made with high precision repeatability and with virtually any design. In contrast, developing tissue-based valves is extremely time consuming and costly as the tissue must be harvested from animals and hand-sutured onto a stent frame leading to high variability in the final product. Additionally, variable thickness leaflets can only be achieved with polymer materials by injection or compression molding with a precisely defined thickness across the entire leaflet surfaces by two mating mold parts, as described herein.

Previous studies have aimed to optimize TAVR stent frames using specific optimization algorithms, with the number of adjustable design parameters limited to only i few in order to keep the optimization within a proper degree of complexity (Claiborne T E, et al., ASAIO journal (American Society for Artificial Internal Organs: 1992). 2013; 59(3):275-83; Barati S, et al., Computers in biology and medicine. 2021; 139:104942; Carbonaro D, et al., Structural and Multidisciplinary Optimization. 2021, 10.1007/s00158-021-02944-w). Optimization of a dynamic complex geometry, such as that represented by the polymeric BAV having a variable thickness leaflets design, requires an approach that departs from conventional engineering optimization methods that typically refer to a total design space, in which some form of optimization algorithm and sensitivity analysis is employed, yet maintaining the hallmarks of a design optimization process. In this study, the goal of the optimization was to converge on an overall polymeric leaflet design that produces improved hemodynanics in a BAV anatomy combined with reduced polymeric leaflet stresses that translate to enhanced durability, which presents a more challenging optimization problem, as compared to a conventional optimization algorithm that would be reasonable for a stent frame design. A conventional optimization approach would not suffice due to the numerous parameters that need to be adjusted (some too complex to define by single values—for example, the shape of the leaflet free edge).

Briefly (and as described above), the systolic orifice area and the von Mises stress magnitudes were used as the global guiding variables, followed by an iterative numerical process that tested numerous design parameters to maximize the geometric orifice area (GOA) and minimize the local and global stress concentrations. Some of these parameters included: leaflet surface area, overall height of the leaflets, leaflet belly length and angle, leaflet free edge length and shape, leaflet attachment shape/angle, coaptation height and shape, location of central coaptation region, and steepness of the commissures. In a subsequent optimization step, the local variable thickness of each leaflet was then optimized as well. This was performed multiple times until stress values plateaued between thickness optimization iterations. After achieving a 20% decrease in the peak stress near the attachment region and additional decrease in stresses in the belly of the leaflet, the leaflets' motion and stresses were finally optimized.

The post-deployment structural analysis and pressure-based flow analysis showed that the AP-BAV offered an increase in GOA and EOA (from 10% up to nearly 50%) at peak systole over the Evolut R. The most eccentric deployments (in patients 1 and 5) benefited from the elliptical shape of the asymmetric polymeric leaflet design, since the leaflets were able to operate closer to their “zero-stress” state, promoting proper function and range of motion (see FIG. 10). Conversely, the Evolut R leaflets, designed for a circular orifice, struggled to open completely with the leaflets stretched in an undesirable manner or excessively folded due to the elliptical orifice. Additionally, the Evolut R leaflets appeared to be stiffer in comparison to the polymeric asymmetric BAV leaflets, even though the elastic modulus of the porcine pericardial tissue is lower than the xSIBS material in the range of the operational strain values. This likely can be attributed to the variable thickness in the polymeric leaflets which provides them with greater flexibility, as compared to the Evolut R tissue leaflets with uniform thickness. Evidently, the larger GOA and EOA achieved by the AP-BAV resulted from its eccentric shape, leaflet configuration, and its optimized variable thickness.

The increased orifice area provided by the AP-BAV will reduce the workload on the heart. Occluded aortic valves require the left ventricle to generate more pressure to drive blood flow and meet the blood supply needs of the body (Carabello B A. Circulation: Cardiovascular Imaging. 2013; 6(6):858-60; Yan W, et al., Frontiers in Physiology. 2021; 12). This can result in the thickening of the left ventricular wall and, over time, potentially cause cardiac hypertrophy or heart failure (Carabello B A. Circulation: Cardiovascular Imaging. 2013; 6(6):858-60; Yan W, et al., Frontiers in Physiology. 2021; 12). Although TAVR intervention can drastically improve the hemodynamics of the stenosed valve, it is typically incapable of achieving an orifice as large as a healthy valve, even with typical manufacturer's recommended valve over-sizing. Slight insufficiencies in terms of the valve performance (EOA, GOA, pressure gradient) that remain after TAVR can still result in poor future outcomes (Barker C M, et al., Cardiac Interventions Today. 2018, (Mitral Considerations):S3-S6). Therefore, the large increase in orifice area achieved by the asymmetrical polymeric leaflets described herein is highly beneficial for long-term efficacy of a BAV-dedicated TAVR device design, as well as the cardiac health of the patient.

The CFD analysis showed superior systolic hemodynamics of the AP-BAV, as compared to the Evolut R valve, with marked differences between the two models for all cases studied. The jet velocity in the Evolut R valve exceeded that of the AP-BAV in the range of 8% to 53%. The lower velocity in the latter is clearly attributed to its larger EOA. The patients with the largest difference in EOA had the largest difference in the jet velocities accordingly, and vice versa. Reducing the velocity through the aortic valve can prevent the formation of unstable jets and flow patterns that could increase the shear stresses on the surrounding walls and the risk of thrombosis.

The difference in magnitude of WSS on the aortic walls between the valves was also significantly large, where the Evolut R produced WSSs between 24% and 103% greater than the AP-BAV. The maximum WSS in patient 5—with a marked 103% increase in WSS—is located in the sinus region adjacent to the leaflets crumpled formation (see FIG. 10), suggesting that a high velocity jet flow likely developed in this area due to the distorted geometry of the leaflets abutting a narrow channel between the stent and aortic wall. In all other cases, the maximum WSS was located at the base of the ascending aorta where the jet flow collides with the wall. Patients 2 and 3 in the Evolut R model had the highest WSS in this location because the velocity in their jet flows was the largest (close to 3 m/s). This elevated velocity was located very close to the wall, creating a local high velocity gradient. In contrast, the AP-BAV deployed in these patients had a large buffer of lower velocities between the aortic wall and the jet flow, thus reducing the velocity gradient and consequently the WSS in this location. The elevated jet velocity in the Evolut R allows the blood to travel along its initial trajectory for longer and collide with the aortic arch before dissipating. This repetitive collision across cardiac cycles will contribute to damaging the endothelial layer and weakening the aortic wall which may lead to the formation of an aneurysm that could rupture or result in aortic dissection (Bollache E, et al., J Thorac Cardiovasc Surg. 2018; 156(6):2112-20.e2; Meierhofer C, et al., European Heart Journal—Cardiovascular Imaging. 2012; 14(8):797-804).

The use of patient-specific BAV models in this study has enabled the testing of a BAV-dedicated valve design in realistic anatomies. Many in silico studies analyzing the aortic valve or TAVR employ idealized patient models (Marom G, et al., Medical & biological engineering & computing. 2013; 51(8):839-48; Lavon K, et al., Medical & biological engineering & computing. 2019; 57(10):2129-43; Ghosh R, et al., Journal of biomechanical engineering. 2018, 10.1115/1.4040600) that are typically designed parametrically with equal sized leaflets and sinuses, or equally distributed angles between the leaflets. These models often fail to capture the complex anatomy of the aortic root, especially in BAV anatomies that exhibit high variability in leaflet size and proportion, raphe location, calcium volume and their deposition patterns, and commissure location. These distinctive features impact the TAVR deployment and hemodynamics. Therefore, using six patient-specific models not only allowed for a unique and relevant comparative study with the Evolut R, but also provided an array of clinical scenarios to test the design and assess its efficacy.

Aside from establishing the design concept for treating BAVs, this study also highlighted the role that structural and fluid simulations can play in the design phase of a device, such as a transcatheter aortic valve. Simulation technology allowed for the design space to be explored before committing to the manufacture of costly prototypes and performance of time-consuming experiments. In this study, numerous iterations of the asymmetric polymeric leaflets were designed before performing and achieving in silico optimization. With this converged design, a prototype can be fabricated and tested for in vitro performance with greater confidence in the device's viability.

### CONCLUSIONS

The Examples relate to the first known design of an asymmetric polymeric leaflets TAVR device which may be used for BAV patients. This device is designed to conform more closely to the elliptical BAV orifice to mitigate the complications of employing current circular TAVR technology, which is not specifically designed for BAV anatomies. The device also implements features such as variable thickness leaflets selected to move, as needed, and reduce the stress magnitudes across the valve. The asymmetric artificial polymeric bicuspid aortic valve (AP-BAV) was compared to Medtronic's Evolut R 29 valve in patient-specific BAV models, and the AP-BAV showed a significant increase in GOA and EOA, as well as a significant decrease in jet velocity and WSS, as compared to the Evolut R. The use of patient-specific anatomies enabled diverse testing conditions for the asymmetric polymeric leaflets.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific instances of the invention described herein. Such equivalents are intended to be encompassed by the following claims.

1. An artificial polymeric valve system comprising:
a supportive frame and a plurality of polymeric leaflets;
wherein the supportive frame comprises a plurality of openings, wherein the supportive frame has a long axis and at least one or more cross-sections taken orthogonally to the long axis have a non-circular shape, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end; and
wherein the polymeric leaflets are able to open and close at an operative end, wherein when the plurality of polymeric leaflets is in the closed position, the operative ends of the leaflets abut each other.

2. The system of claim 1, wherein the non-circular shape is selected from elliptical, lemniscate, cardioid, quartic bean curve, or polygonal; or the non-circular shape comprises fluctuating asymmetry; or the non-circular shape is a copy of the cross-sectional shape of a native valve of a subject, and optionally the native valve of the subject is a bicuspid aortic valve.

3. An artificial polymeric valve system comprising:
a supportive frame and a plurality of polymeric leaflets;
wherein the supportive frame comprises a plurality of openings, and wherein the supportive frame is embedded in a polymeric matrix in the form of a polymeric sleeve;
wherein each of the polymeric leaflets in the plurality is a continuum of the polymeric sleeve without sutures, and each of polymeric leaflets is connected to the polymeric sleeve at an attachment end;
wherein at least one or more of the plurality of polymeric leaflets is asymmetrical to the other polymeric leaflets in the plurality; and
wherein the polymeric leaflets are able to open and close at an operative end, wherein when the plurality of polymeric leaflets is in the closed position, the operative ends of the leaflets abut each other.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. The system of claim 1, wherein the polymeric sleeve comprises one or more polymers selected from the group consisting of thermoplastic polymers/elastomers, thermoset polymers, thermally crosslinkable polymers, elastomeric polymer biomaterials, polymers containing polysulfone(s), PTFE, crosslinked poly(styrene-isobutylenese-styrene) (xSIBS), poly(styrene-isobutylene-styrene) (SIBS), polymyrcene, polymenthide, and poly(F-decalactone), silicones, polyolefins, polydiene elastomers, poly(vinyl chloride), natural rubbers, heparinized polymers, hydrogels, polypeptides elastomers, polysiloxane-urea elastomers, and polyurethanes.

16. The system of claim 1, wherein the polymeric sleeve further comprises one or more anti-leak flaps thereon.

17. (canceled)

18. (canceled)

19. The system of claim 1, wherein each of the polymeric leaflets in the plurality comprises variable thickness having a center line of symmetry, wherein a given cross-section each of the leaflets has at least two or more thicknesses.

20. The system of claim 19, wherein each of the leaflets has a minimum thickness in a range from about 50 to 200 μm or in a range from about 200 to 600 μm.

21. (canceled)

22. (canceled)

23. (canceled)

24. A method of making an artificial polymeric valve system, the method comprising the steps of:
(i) deforming a supportive frame, wherein the supportive frame has a long axis and at least one or more cross-sections taken orthogonally to the long axis have a non-circular shape after the supportive frame is deformed;
(ii) placing the deformed supportive frame inside a cavity of a mold in a position suitable to facilitate flow of the polymer around the deformed supportive frame,
wherein the mold comprises a top core, a bottom core, and one or more encasement components which encase the top and bottom cores,
wherein the top core and the bottom core define a shape to form a plurality of polymeric leaflets in a suitable orientation wherein the plurality of polymeric leaflets are formed optionally in a semi-open position having a zero residual stress conformation,
optionally, wherein the shape forming the plurality of polymeric leaflets has a curvilinear profile along a circumferential axis of the polymeric leaflets being formed; and
(iii) introducing at least one polymer into the mold;
(iv) molding or casting the plurality of polymeric leaflets and a polymeric sleeve around the deformed supportive frame to form the artificial polymeric valve system,
wherein the plurality of polymeric leaflets and the polymeric sleeve are a continuum and each of the polymeric leaflets are connected to the polymeric sleeve at an attachment end; and
(v) removing the artificial polymeric valve system formed from the mold.

25. The method of claim 24, wherein the non-circular shape is selected from elliptical, lemniscate, cardioid, quartic bean curve, or polygonal; or the non-circular shape comprises fluctuating asymmetry; or the non-circular shape is a copy of the cross-sectional of a shape of a native valve of a subject, and optionally the native valve of the subject is a bicuspid aortic valve.

26. The method of claim 24, wherein the number of polymeric leaflets formed in step (iv) is two, three, four, five, six, seven, eight, nine, ten, eleven, or twelve polymeric leaflets.

27. (canceled)

28. (canceled)

29. The method of claim 24, wherein at least one of the polymeric leaflets present is not symmetrical with the other polymeric leaflets in the plurality.

30. (canceled)

31. (canceled)

32. (canceled)

33. (canceled)

34. (canceled)

35. (canceled)

36. (canceled)

37. (canceled)

38. (canceled)

39. The method of claim 24, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.

40. The method of claim 24, wherein the at least one polymer is selected from one or more of the group consisting of thermoplastic polymers/elastomers, thermoset polymers, thermally crosslinkable polymers, elastomeric polymer biomaterials, polymers containing polysulfone(s), PTFE, crosslinked poly(styrene-isobutylenese-styrene) (xSIBS), poly(styrene-isobutylene-styrene) (SIBS), polymyrcene, polymenthide, and poly(F-decalactone), silicones, polyolefins, polydiene elastomers, poly(vinyl chloride), natural rubbers, heparinized polymers, hydrogels, polypeptides elastomers, polysiloxane-urea elastomers, and polyurethanes.

41. The method of claim 24, wherein the polymeric sleeve further comprises one or more anti-leak flaps thereon.

42. The method of claim 24, wherein the method further comprises a step of coating the polymeric sleeve, after step (v) with a gel paving material onto one or more outer surfaces of the polymeric sleeve.

43. The method of claim 24, wherein each of the plurality of polymeric leaflets comprises variable thickness across cross-sections of each of the polymeric leaflets running from an attachment end to an operative end thereof.

44. The method of claim 43, wherein each of the plurality of polymeric leaflets has a minimum thickness in a range from about 50 to 200 μm or in a range from about 200 to 600 μm.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A method of replacing a defective valve in a subject in need thereof, the method comprising the steps of:
(a) inserting an artificial polymeric valve system of claim 1 in a crimped, collapsed, or compacted state into the subject in need thereof;
(b) delivering the artificial polymeric valve system to the defective valve in the subject in need thereof;
(c) implanting the artificial polymeric valve system by expanding the crimped, collapsed, or compacted artificial polymeric valve system into an expanded state, which localizes or fixes the artificial polymeric valve system at the defective valve and replaces the function of the defective valve.

69. The method of claim 68, wherein the defective valve is vascular valve, pulmonary valve, or urinary valve.

70. The method of claim 69, wherein the vascular valve is a venous valve or a cardiac valve; optionally wherein the cardiac valve is selected from the group consisting of aortic valve, mitral valve, bicuspid aortic valve, tricuspid aortic valve, or pulmonary valve.

71. (canceled)

72. (canceled)

73. (canceled)

74. (canceled)

75. (canceled)
